(19) United States
(12) Patent Application Publication
(10) Pub. No.: US 2015/0079052 A1
(43) Pub. Date: Mar. 19, 2015

(54) Methods for amelioration of autoimmune disease using stem cells transduced with T cell receptors from IL-10 secreting T cells

(71) Applicant: PRESIDENT AND FELLOWS OF HARVARD COLLEGE, CAMBRIDGE, MA (US)

(72) Inventors: Jack L. Stominger, Cambridge, MA (US); Sze-Ling Ng, Somerville, MA (US)

(21) Appl. No.: 14/501,446

(22) Filed: Sep. 30, 2014

Related U.S. Application Data
(63) Continuation of application No. PCT/US2013/032660, filed on Mar. 15, 2013.
(60) Provisional application No. 61/618,015, filed on Mar. 30, 2012.

(51) Int. Cl. A61K 35/28; C12N 15/86

(52) U.S. Cl. CPC A61K 35/28; C12N 15/86; C12N 2740/10041
USPC 424/93.21; 435/320.1; 435/325

(57) ABSTRACT

Methods, cells and vectors are provided for treating a subject for an autoimmune disease using stem cells transduced with genes encoding T cell receptors from IL-10 secreting regulatory T cells. The IL-10 secreting regulatory T cells are obtained from a donor of stem cells, the donor having been immunized with peptidic compositions used in the treatment of the autoimmune disease.

### RELATED APPLICATIONS

The present application claims the benefit of U.S. provisional application Ser. No. 61/618,015 filed in the U.S. Patent and Trademark Office Mar. 30, 2012 and PCT/US13/32660 filed in the PCT Receiving Office of the U.S. Patent and Trademark Office Mar. 15, 2013 entitled “Methods for amelioration of autoimmune disease using stem cells transduced with T cell receptors from IL-10 secreting T cells”, inventors Sze-Ling Ng and Jack L Strominger, each of which is hereby incorporated by reference herein in its entirety.

### TECHNICAL FIELD

Methods, cells and vectors are provided for amelioration of autoimmune diseases using stem cells transduced with genes encoding T cell receptors from IL-10 secreting T cells.

### BACKGROUND

Millions of people worldwide suffer from autoimmune diseases. Over 80 autoimmune diseases are known, including multiple sclerosis, rheumatoid arthritis, psoriasis, lupus, Crohn's disease and type I diabetes. Multiple sclerosis (MS) affects about one in a thousand in Western populations and is thought to be due to an expansion of T cells autoreactive to a myelin protein, e.g., myelin basic protein (MBP), phospholipid protein (PLP), or myelin oligodendrocyte protein (MOG). (Stern et al. 2008 Proc. Natl. Acad. Sci. USA 105:5172-5176). An animal model of MS is experimental autoimmune encephalomyelitis (EAE), which is induced in appropriate strains of mice or rats by immunization with an MBP, PLP or MOG protein or peptide derived from one of these proteins.

Cells that play a role in the regulation of immune response include regulatory T cells. Several autoimmune diseases including both multiple sclerosis and diabetes are associated with a deficit in the number or function of regulatory T cells. (Astier, A. L. and Hafler, D. A. 2007, J. Neuroimmunol. 191(1-2):70-78). T regulatory cells of patients with MS and of Cynomolgus monkeys with induced disease are defective. (Costantino, C. M. et al. 2008. J Clin Immunol. 28(6):697-706; Astier, A. L. and Hafler, D. A. 2007, J. Neuroimmunol. 191(1-2):70-78; M A et al. 2009, Int. Immunopharmacol. 9(5):599-608).

Drugs used to treat autoimmune diseases do not selectively target immune cells and frequently have severe side effects, such as nephrotoxicity and neurotoxicity. Thus, there is an unmet need for safer and more effective immunosuppressant therapies for autoimmune and inflammatory diseases and also for organ transplant rejection.

### SUMMARY

Approaches using expansion of regulatory T cells have the potential for providing targeted and safer methods of treatment of autoimmune diseases and conditions. Accordingly methods are described herein for increasing the ability of regulatory T cells to reduce or eliminate symptoms of an autoimmune disease, for example experimental autoimmune encephalomyelitis, the mouse model of the human disease multiple sclerosis. Transducing genes encoding the T cell receptor α and β pair polypeptides from a regulatory T cell line expressing one or more cytokines at a high level into a retroviral vector, followed by transducing mouse hematopoietic stem and progenitor cells with the retroviral vector, and transplanting or grafting the transduced cells into murine subjects generates retrogenic mice, that express high frequency of regulatory T cells having a similar cytokine expression profile. The regulatory T cells lines described herein are inducible by peptidic compositions such as amino acid copolymers used in the treatment of both experimental autoimmune encephalomyelitis and multiple sclerosis, and secrete interleukin 10 (IL-10). Methods described herein are based on the fact that a set of T cell receptor α and β pair chains i.e. polypeptides from an induced regulatory T cell line is associated with information that results in IL-10 secretion by regulatory T cells originating from the transplanted hematopoietic stem or progenitor cells. Retrogenic mice with increased frequency of regulatory T cells having a particular set of T cell receptor α and β pair polypeptides, and secreting a high level of a desired cytokine, are useful in affecting amelioration or preventing induction of an autoimmune disease and development of therapies for treatment of autoimmune diseases in humans.

An embodiment of the invention herein is a method for treating a subject for an autoimmune disease, including: isolating bone marrow cells from a donor subject and enriching the cells for the presence of hematopoietic stem and progenitor cells (HSC/HPC) and transducing the HSC/HPC by a retroviral vector containing a heterologous gene encoding a set of T cell receptor α and β pair polypeptides linked by an in vivo cleavable peptide sequence, such that the set of T cell receptor α and β pair polypeptides is obtained from a regulatory T cell line that secretes a high level of at least one cytokine upon stimulation with a peptidic composition that binds to the set of T cell receptor α and β pair polypeptides; conditioning the recipient for transplantation, and transplanting the transduced HSC/HPC and non-transduced bone marrow cells into the recipient subject having the autoimmune disease; and, measuring a reduction or an elimination of at least one symptom of the autoimmune disease in the recipient subject, thereby treating the recipient subject for the autoimmune disease.

The peptidic composition in various embodiments of the method is selected from the group of: an oligopeptide, a polypeptide, a protein, a protein fragment, and a random amino-acid copolymer.

In related embodiments of the method, the donor and the recipient subjects are the same subject. Alternatively, the donor and the recipient subjects are different subjects.

In various embodiments of the method, conditioning for transplantation includes irradiating the recipient subject. Alternatively, conditioning for transplantation includes administering at least one cytotoxic agent to the recipient.

In related embodiments of the method, the subject is an experimental non-human animal and the autoimmune disease includes experimental allergic encephalomyelitis (EAE), and the method further includes inducing EAE in the recipient after transplantation.

The recipient subject in various embodiments of the method is a human, and the autoimmune disease is selected from: multiple sclerosis, autoimmune hemolytic anemia, autoimmune oophoritis, autoimmune thyroiditis, autoimmune uveoretinitis, Crohn's disease, chronic immune thrombocytopenic purpura, colitis, contact sensitivity disease, diabetes mellitus, Grave's disease, Guillain-Barre's syndrome, Hashimoto's disease, idiopathic myxedema, multiple sclerosis, myasthenia gravis, psoriasis, pemphigus vulgaris, rheumatoid arthritis, and systemic lupus erythematosus.

In related embodiments of the method, the regulatory T cell line secretes a high level of the cytokine interleukin 10 (IL-10) upon stimulation with a peptidic composition including random amino acid copolymer poly (Y,F,A,K)ₙ, also designated YFAK or FYAK.

The set of T cell receptor α and β pair polypeptides in various embodiments of the method includes canonical T cell receptor (TCR) variable regions Vα3.2 and Vβ14. For example, the variable region Vα3.2 of the T cell receptor α polypeptide includes complementarity determining region (CDR) region having the amino acid sequence TTSSGQKLV (SEQ ID NO: 1) and the Jα region Jα16, and the variable region V β14 of the T cell receptor β polypeptide includes CDR region having the amino acid sequence LGGWAEQF (SEQ ID NO: 2) and the Jβ region Jβ2.1. In further examples, the variable region Vα3.2 of the T cell receptor α polypeptide includes CDR region having the amino acid sequence SSSGSWQLI (SEQ ID NO: 3) and the Jα region Jα22, and the variable region V β14 of the T cell receptor β polypeptide includes CDR region having the amino acid sequence PGQYEQY (SEQ ID NO: 4) and the Jβ region Jβ2.7.

In related embodiments of the method, the set of T cell receptor α and β pair polypeptides includes canonical TCR variable regions Vα17 and Vβ4, respectively.

Another embodiment of the invention is a retroviral vector including a nucleotide sequence encoding a set of T cell receptor α and β pair polypeptides linked by a nucleotide sequence encoding an in vivo cleavable peptide, such that the nucleotide sequences of the set of T cell receptor α and β pair polypeptides are obtained from a regulatory T cell line that secretes a high level of at least one cytokine upon stimulation with a peptidic composition that binds to the set of T cell receptor α and β pair polypeptides.

In related embodiments of the retroviral vector, the regulatory T cell line, from which the set of T cell receptor α and β pair polypeptides are derived, secretes a high level of IL-10 upon binding random amino acid copolymer poly (Y,F,A,K)ₙ.

In various embodiments of the retroviral vector the cleavable peptide comprises porcine teschovirus-1 (P2A) peptide having amino acid sequence ATNFSLLKQAGDVEENPGP (SEQ ID NO: 5).

In related embodiments the retroviral vector further includes a nucleotide sequence encoding green fluorescence protein (GFP), such that the GFP encoding nucleotide sequence is located downstream of the nucleotide sequences encoding the set of T cell receptor α and β pair polypeptides and separated by an internal ribosome entry site (IRES) nucleotide sequence.

The retroviral vector, in various embodiments, for example, includes the cleavable peptide, porcine teschovirus-1 (P2A) peptide having amino acid sequence ATNFSLLKQAGDVEENPGP (SEQ ID NO: 5), and the set of T cell receptor α and β pair polypeptides includes canonical TCR variable regions Vα3.2 and Vβ14, respectively. For example, the canonical TCTR variable region Vα3.2 of the T cell receptor α polypeptide includes CDR region having the amino acid sequence SSSGSWQLI (SEQ ID NO: 3) and the Jα region Jα22, and such that the canonical TCR variable region V β14 of the T cell receptor β polypeptide includes a CDR region having the amino acid sequence PGQYEQY (SEQ ID NO: 4) and the Jβ region Jβ2.7. In another example of the retroviral vector the canonical TCR variable region Vα3.2 of the T cell receptor α polypeptide includes CDR region having the amino acid sequence SSSGSWQLI (SEQ ID NO: 3) and the Jα region Jα22, and the canonical TCR variable region V β14 of the T cell receptor β polypeptide includes CDR region having the amino acid sequence PGQYEQY (SEQ ID NO: 4) and the Jβ region Jβ2.7.

In related embodiments, of the retroviral vector, the set of T cell receptor α and β pair polypeptides includes canonical TCR variable regions Vα17 and Vβ4, respectively.

Another embodiment of the invention is a population of transduced hematopoietic stem cells and progenitor cells suitable for engrafting in a host for selectively expressing a set of desired T cell receptor α and β pair polypeptides including: a population of hematopoietic stem and progenitor cells transduced with a nucleotide sequence encoding the set of T cell receptor α and β pair polypeptides linked by a nucleotide sequence encoding an in vivo cleavable peptide, such that the nucleotide sequence encoding the set of T cell receptor α and β pair polypeptides is obtained from a T regulatory cell clone that secretes a high level of at least one cytokine upon stimulation with a peptidic composition that binds to the T cell receptor α and β pair polypeptides.

The peptidic composition, in related embodiments of the population of transduced hematopoietic stem and progenitor cells includes random amino acid copolymer poly (Y,F,A,K)ₙ. In related embodiments of the population of cells, the cytokine secreted at a high level by the regulatory T cell line upon stimulation with the random amino acid copolymer poly (Y,F,A,K)ₙ is IL-10.

In related embodiments of the population of transduced hematopoietic stem and progenitor cells, the T cell receptor α and β pair polypeptides include canonical TCR variable regions Vα3.2 and Vβ14, respectively. For example, the variable region Vα3.2 of the T cell receptor α polypeptide includes CDR region having the amino acid sequence TTSSGQKLV (SEQ ID NO: 1) and the Jα region Jα16, and such that the variable region V β14 of the T cell receptor β polypeptide includes CDR region having the amino acid sequence LGGWAEQF (SEQ ID NO: 2) and the Jβ region Jβ2.1. In a further example, the variable region Vα3.2 of the T cell receptor α polypeptide includes CDR region having the amino acid sequence SSSGSWQLI (SEQ ID NO: 3) and the Jα region Jα22, and such that the variable region V β14 of the T cell receptor β polypeptide includes a CDR region having the amino acid sequence PGQYEQY (SEQ ID NO: 4) and the Jβ region Jβ2.7.

In a related embodiment of the population of transduced hematopoietic stem and progenitor cells, the set of T cell receptor α and β pair polypeptides include canonical TCR variable regions Vα17 and Vβ4.

A related embodiment of the invention is a method for treating a subject for an autoimmune disease by administering an expanded population of regulatory T cells, including: isolating bone marrow cells from a donor subject and enriching the cells for presence of hematopoietic stem cells and progenitor cells (HSC/HPC) and transducing the HSC/HPC with a retroviral vector containing a heterologous gene encoding a set of T cell receptor α and β pair polypeptides linked by an in vivo cleavable peptide sequence, wherein the set of T cell receptor α and β pair polypeptides is encoded by a nucleotide sequences obtained from a regulatory T cell line that secretes a high level of at least one cytokine upon stimulation with a peptidic composition that binds to the T cell receptor α and β pair polypeptides; transplanting the transduced HSC/HPC and non-transduced bone marrow cells into a recipient subject having the autoimmune disease wherein the recipient is conditioned for transplantation; and, measuring a reduction or an elimination of at least one symptom of the autoimmune disease in the recipient subject, thereby treating the recipient subject for the autoimmune disease.

In various embodiments the method further includes after transplanting the transduced HSC/HPC and non-transduced bone marrow cells into the recipient subject having the autoimmune disease and conditioned for transplantation, measuring in the recipient expansion of regulatory T cells expressing the transduced heterologous gene encoding the set of T cell receptor α and β pair polypeptides.

### BRIEF DESCRIPTION OF THE DRAWINGS

FIG. 1 is a list of amino acid sequences of complementarity determining regions (CDRs) of T cell receptor (TCR) α and β chain genes of 18 individual FYAK specific T cells. The TCR pairs marked with an * were used in examples herein for transduction of hematopoietic stem and progenitors cells using a retroviral vector. All TCR pairs have canonical Vα3.2 and Vβ14 genes except the TCR-αβ pair number 16, which uses Vβ 3.5.

FIG. 2 is a schematic diagram of retroviral vector pMIGII (Addgene, Cambridge, Mass.) which expresses a protein of interest and GFP, having nucleotide sequences corresponding to IRES GFP (internal ribosomal entry site green fluorescence protein). The TCR α and β chain genes were cloned upstream of IRES, and the α and β chain genes are separated by porcine virus 2A peptide sequence that is cleaved spontaneously in vivo to produce equal amounts of TCR α and TCR β proteins that assemble into a TCR.

FIG. 3 is a set of FACS profiles of expression of TCR αβ pair on the surface of transfected HEK 293T cells analyzed by monoclonal antibodies specific to TCR Vα3.2 and TCR Vβ14.

FIG. 4 is a bar graph showing amount of cytokine secretion by T cells derived from bone marrow hematopoietic progenitor cells transduced with TCR from IL-10 secreting regulatory T cells, and developed in vitro on OP9-DL1 cells. TCR1-4 are identified by asterisks (*) in FIG. 1. Each group of bars in the graph arranged from left to right shows data from of cells transduced with: vector alone, TCR1, TCR2, TCR3 or TCR4.

FIGS. 5A, and 5B are a set of plots of FACS analysis of tail blood from retrogenic mice.

FIG. 5A is a set of FACS dot plots of cells from the peripheral blood of vector control, TCR αβ pair 1 (TCR1) and TCR αβ pair 7 (TCR2) retrogenic mice. TCR αβ pair numbering is according to FIG. 1. CD4 and GFP amounts are shown along the Y and X axes respectively.

FIG. 5B is a set of FACS histograms of profiles of cells from the peripheral blood of vector control, TCR αβ pair 1 (TCR1) and TCR αβ pair 7 (TCR2) retrogenic mice showing amounts of Vβ14 TCR.

FIGS. 6A, 6B and 6C are a set of bar graphs of cytokine secretion profiles of TCR transduced cells and control cells transduced by parent (empty vector).

FIG. 6A is a bar graph of secretion of IL-10 measured by Enzyme-linked immunosorbent assay (ELISA) of supernatants from YFAK-stimulated CD4+ GFP+ TCR1, TCR2 expressing FACS sorted splenic T cells from retrogenic mice.

FIG. 6B is a bar graph of analysis of anti-inflammatory cytokines IL-10 and IL-5 and pro-inflammatory cytokines IL-1β and TNFα from supernatants of YFAK-treated CD4+ purified TCR2 expressing GFP+ FACS sorted splenic T cells, compared with similarly treated control cells obtained from retrogenic mice transplanted with vector transduced cells. The analysis was performed using the Bioplex system (BioRad, Hercules Calif.).

FIG. 6C is a bar graph of analyses (using Bioplex system) of anti-inflammatory cytokines IL-10 and IL-5 and pro-inflammatory cytokines IL-1β and TNFα from supernatants of YFAK-treated CD4+ purified TCR1 expressing GFP+ FACS sorted splenic T cells, compared with similarly treated control cells obtained from retrogenic mice transplanted with vector transduced cells. TCR1 and TCR2 differ only in their CDR3 regions (FIG. 1).

FIG. 7 is a graph of clinical scores of EAE in SJL retrogenic mice that were transplanted with HSC/HPC transduced with TCR Vβ14 Vα3.2 as a function of time post EAE induction. The retrogenic mice transplanted with HSC/HPC transduced with TCR Vβ14 Vα3.2 were observed to be protected from EAE as determined by a decrease in clinical score. Vector control retrogenic and TCR retrogenic mice were injected subcutaneously with 100 ug of PLP139-151 emulsified in CFA at day 0 to induce EAE when the retrogenic mice were 16 weeks old. Mice were scored daily, and the mean score for four vector retrogenic mice and five TCR retrogenic mice were plotted. The mean maximal scores of individual mice were 3, 3, 5, and 5 for retrogenic vector mice and 0, 2, 1, 0, and 0 for TCR retrogenic mice.

FIG. 8 is an exemplary nucleotide sequence of a retroviral vector pMIGII (SEQ ID NO: 6) used in examples herein for inserting a nucleotide sequence of a fusion polypeptide having a set of T cell receptor α and β pair polypeptides derived from an inducible regulatory T cell line that secretes a high level of at least one cytokine upon stimulation with a peptidic composition that binds to the set of T cell receptor α and β pair polypeptides expressed by the regulatory T cell line.

FIG. 9 is a set of amino acid sequences: T cell receptor variable α3.2 region (Vα 3.2), SEQ ID NO: 7; T cell receptor constant α region, SEQ ID NO: 8; T cell receptor variable 314 region (Vβ14), SEQ ID NO: 9; and, T cell receptor constant β2 region SEQ ID NO: 10. The T cell receptor α and β amino acid sequences, with variations in the CDR and J regions within these sequences were used in construction of retroviral vectors for transduction of hematopoietic stem cells and progenitor cells as described in Examples herein.

FIG. 10 is a set of exemplary nucleotide sequences TCR1, TCR2, TCR3 and TCR4 (SEQ ID NOs: 11, 12, 13 and 14, respectively), each encoding a non-identical T cell receptor α and β pair polypeptide linked by an in vivo cleavable porcine teschovirus-1 (P2A) nucleotide sequence. The T cell receptor α and β pair polypeptides each have a T cell receptor variable α 3.2 region (Vα 3.2) and T cell receptor variable β14 region (Vβ 14). Each of the sequences shown differs in sequences of the CDR and J regions.

FIG. 11 is a set of two bar graphs showing amount of secretion of the cytokines IL-10 and IL-5 by T cells from retrogenic mice transplanted with empty vector, TCR1 or TCR2 transduced hematopoietic stem and progenitor cells upon stimulation with different peptidic compositions. Secretion of IL-10 and IL-5 were observed to follow the same pattern. Cells derived from mice transplanted with empty vector transduced HPS/HPC produced IL-10 and IL-5 only when stimulated with PMA. Cells derived from mice transplanted with TCR2 transduced HPS/HPC produced high amounts of IL-10 and IL-5 only when stimulated with FYAK, and to a lesser extent when stimulated with YEAK.

FIGS. 12A, 12B, 12C and 12D are a set of graphs of disease score as a function of time after induction of EAE in retrogenic mice transplanted with hematopoietic stem and progenitor cells transduced with either the empty vector or TCR1. Higher score numbers reflect increased severity of the disease: 0 (no change in motor function); 1 (limp tail); 2 (limp tail and weakness of hind leg); 3 (limp tail and hind leg paralysis); 4 (limp tail, hind leg paralysis, partial front leg paralysis; and 5 (complete paralysis or death).

FIG. 12A is a graph of disease score as a function of time for an example in which EAE was induced when mice were 8 weeks old. Protection was observed in mice transplanted with hematopoietic stem and progenitor cells transduced with TCR1.

FIG. 12B is a graph of disease score as a function of time for an example in which EAE was induced when mice were 9 weeks old. Better protection from EAE was observed if a longer period of recovery is allowed after transplantation.

FIG. 12C is a graph of disease score as a function of time for another example in which EAE was induced when mice were 8 weeks old. Higher death was observed in mice transplanted with hematopoietic stem and progenitor cells transduced with TCR1.

FIG. 12D is a graph of disease score as a function of time for another example in which EAE was induced when mice were 9 weeks old. Lower overall death was observed if a longer period of recovery was allowed after transplantation.

FIG. 13 is a list of amino acid sequences at the ends of Vβ and Vα Framework region 3.

FIG. 14 illustrates stapling peptides using the regulatory J5 peptide 15 mer as an example. Binding of J5 peptide 15 mer to I-A^{s} (left) and its modification by hydrocarbon stapling (right) is shown. Upward pointing residues are identified by asterisks (*). Anchor residues are identified by closed circles (). In the helix I, i+3, the D-(R-) configuration at P5 with the L-(S-) configuration at P8 may be the best.

### DETAILED DESCRIPTION OF THE EMBODIMENTS

The random amino acid copolymer poly(Y,F,A,K)ₙ (YFAK or FYAK), and less efficiently poly(Y,E,A,K)n (YEAK, also known as Copaxone, Copolymer 1 and glatiramer acetate), induce differentiation of regulatory T cells that secrete principally the immunomodulatory cytokine interleukin-10 (IL-10) principally, and other cytokines in smaller amounts (Stern, J. N. et al. Proc Natl Acad Sci USA, 2008. 105(13): 5172-76). IL-10 secreting regulatory cells mediate bystander immunosuppression and lead to amelioration of Experimental Autoimmune Encephalomyelitis (EAE), the murine model of the human disease Multiple Sclerosis (Stern et al. 2008). YEAK is a drug widely used in the therapy of multiple sclerosis. YFAK-specific T cell lines have been generated and the nature of the T cell receptors expressed by these cell lines have been investigated (Zhang, H., et al. Proc Natl Acad Sci USA 2009 106(9): 3336-41). T cell receptor is composed of alpha and beta chains, containing variable and constant regions. Interestingly, YFAK-induced regulatory T cells preferentially used Vα3.2 with Vβ14, and engagement of their TCR led to the production of a high level of IL-10 (Zhang et al. 2009).

Immunization with certain TCR derived peptides provided protection against EAE and induced Tregs (Madakamutil, L. T. et al. J Immunol 2008. 180(7): p. 4577-85; Vandenbark, A. A. et al. J Neurosci Res 1996. 43(4): p. 391-402; Kumar, V. et al. J Immunol 1995. 154(4): p. 1941-50; and Bourdette, D. N. et al. Mult Scler 2005. 11(5): p. 552-61). Analogous to immunization with the J5 peptide 15-mer which allows the generation of IL-10 secreting T-cells (Stern, J. N. Proc Natl Acad Sci USA, 2005. 102(5): 1620-25), some peptides within the sequence of the TCR may be responsible for the induction of selective IL-10 secretion by these regulatory T cells. However, the regulatory T cells generated have not yet been fully characterized. A cytokine secretion profile of an uncharacterized T cell line showed that IFN-γ was the main cytokine secreted and very little IL-10 was detected (Kumar, V. et al. J Immunol, 1998. 161(12): p. 6585-91). Somatic cell nuclear transfer (SCNT) from T cells that secrete IFN-γ into embryonic stem (ES) cells that results in a reprogramming the transferred nuclei and removal of epigenetic marks has been reported (Kirak, O., et al. Science, 2010. 328(5975): p. 243-8). These ES cells were used to generate mice that developed T cells that secreted IFN-γ. This result raises the possibility that the TCR genes of the T cells used for SCNT may provide the information for IFN-γ secretion, although it remains possible that all epigenetic marks were not reprogrammed in the ES cell. In another example utilizing H-2^{u} mice, some feature of the TCR was postulated to be important in inducing tolerance to a myelin basic protein peptide (Perchellet, A., et al. Nat Immunol, 2004. 5(6):606-14.). Thus some structural feature of the TCR itself may be important in generating regulatory T cells that have a specific cytokine secretion profile, and therefore T cell phenotype.

Retrogenic T cells have been generated in vitro and in vivo in SJL mice after infection of bone marrow-derived hematopoetic stem and progenitor cells (HSC/HPC) with retroviruses encoding both the α and β chains of TCR from the copolymer-specific, IL-10-secreting regulatory T cells. To reach this goal the nature of the T cell receptors in YFAK-specific cells generated by immunizing SJL mice with YFAK and by restimulation were characterized (Zhang, H., et al. Proc Natl Acad Sci USA 2009 106(9): 3336-41). TCR Vβ and TCR Vα usage were found to be oligoclonal. The principal TCR Vβ segments utilized were Vβ14 (30%) and Vβ4 (24%) as determined by using a panel of Vβ-specific monoclonal antibodies. Analysis of Vα segment was performed by PCR since few Vα-specific monoclonal antibodies are available and the data obtained showed preferential usage of Vα3.2 with the Vβ14 subunit.

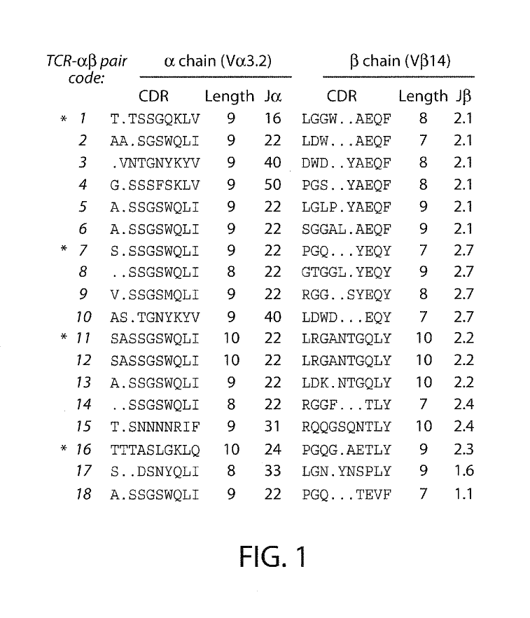

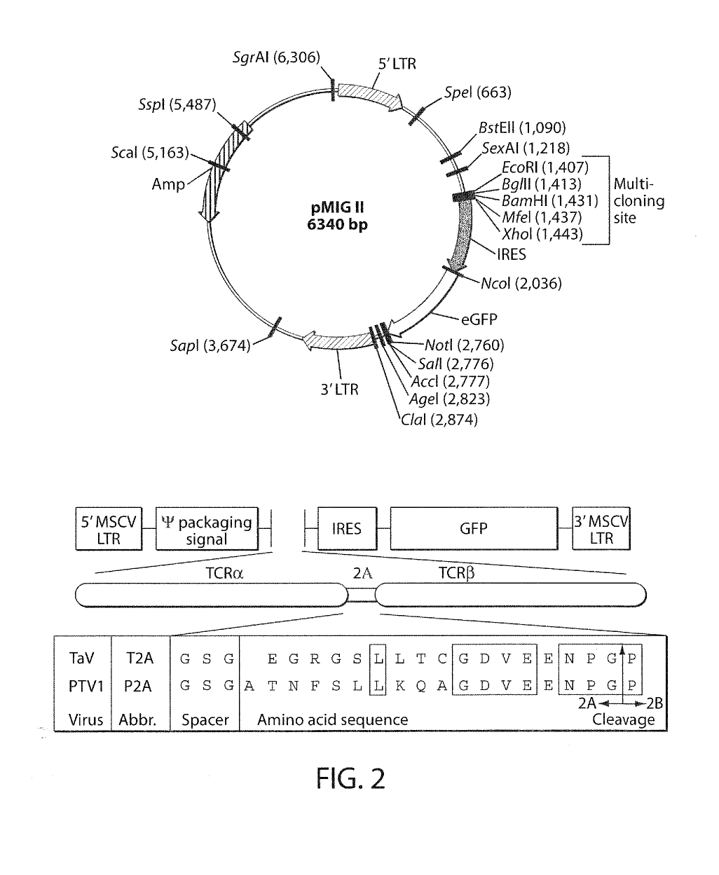

Single cell RT-PCR was used herein to obtain 18 full-length TCRαβ pair cDNAs that utilized Vβ14 and Vα3.2 (one of eight subtypes of Vα3, one a chain was Vα3.5 rather than Vα3.2) (Zhang et al. 2009). The CDR3 sequences were well conserved in the case of Vα but much more diverse in the case of Vβ (FIG. 1). Pairs marked with asterisks representing reasonably diverse CDR3 sequences and were selected for further analysis. For further examples, these cDNA pairs were inserted into a retrovirus vector from which green fluorescence protein (GFP) is also expressed to generate a construct expressing the TCR α and β chain proteins. The TCR α and β chain cDNAs were separated by the porcine virus 2A peptide sequence that cleaves spontaneously in vivo to yield in equal amounts separated TCRα and TCRβ proteins that assemble into a TCR (Holst, J. et al. Nature Methods (2006) 3: 191-197; Hoist, J. et al. Nature Protocols (2006) 1: 406-417; FIG. 2).

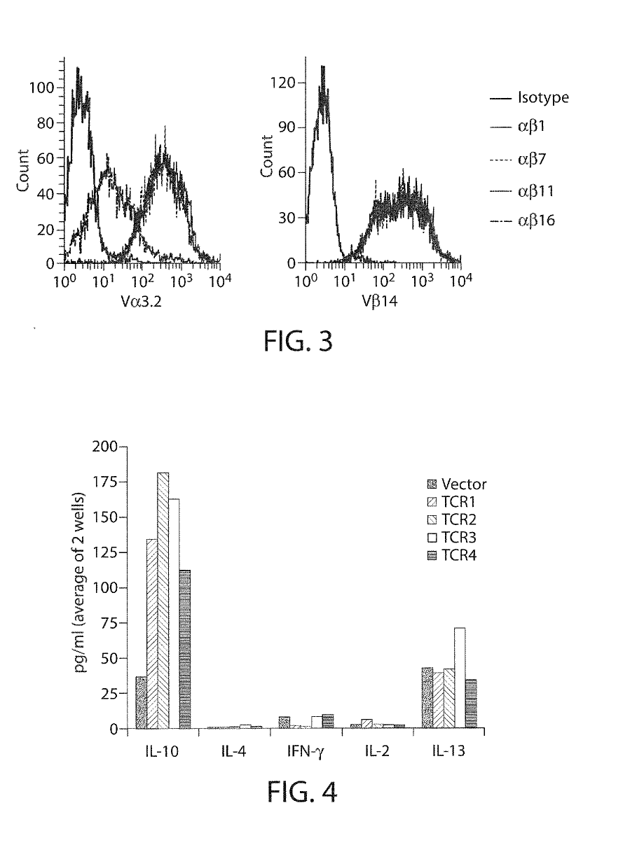

Transfection of HEK 293T cells resulted in surface expression of TCR αβ pairs as shown by reactivity with Vα3.2 and Vβ14 specific monoclonal antibodies (FIG. 3). Infection of two different T cell hybridomas from which the TCRα and TCRβ genes had been deleted (TCRα-/β-hybridomas), resulted in surface expression of a TCR in about 50% of the hybridoma cells. The TCRα-β-hybridomas used had the epigenetic change that programs the cells for IL-2 secretion. Thus, IL-2 secretion from these hybridomas was induced specifically by YFAK, the immunizing copolymer, indicating that the TCRαβ pairs introduced by the retroviruses were functional (Zhang et al., Ibid., 2009).

Immunization with peptides derived from the framework and other regions of TCR, most recently from the CDR3 regions, has been known to protect from EAE and to induce regulatory T cells, including preferential usage of Vβ14 and a peptide derived from it (Madakamutil, L. T. et al. J Immunol 2008. 180(7): p. 4577-85; Vandenbark, A. A. et al. J Neurosci Res 1996. 43(4): p. 391-402; Kumar, V. et al. J Immunol 1995. 154(4): p. 1941-50; and Bourdette, D. N. et al. Mult Scler 2005. 11(5): p. 552-61; Kumar, V. et al. J Immunol 1998. 161(12): p. 6585-91.). The results above of the functional property TCR αβ pair protein obtained from introduction of TCR α and β chain genes with the data of immunization of TCR derived peptides indicated that some structural feature of the TCR itself is important in generating IL-10-secreting regulatory T cells that induce tolerance.

The phrase “retrogenic mice” as used herein refers to mice generated using the retrogenic approach (‘retro’ from retrovirus and ‘genic’ from transgenic), which provides an alternative method to a transgenic mouse approach for expressing selected T cell receptor genes in the T cells of an engineered mice. The retrogenic approach uses an in vivo cleavable peptide linked multicistronic retroviral vector for transferring genes to stem or progenitor cells, which cells are subsequently transplanted into a host. (Holst J. et al. 2006, Nat Protoc, 1(1):406-417; Holst J. et al. 2006, Nat Methods, 3(3):191-197). Upon engraftment in the host, the cells that develop from the differentiation of the transplanted stem or progenitor cells express the transferred genes and produce the corresponding proteins. Examples herein show increased secretion (e.g., at least about 10 fold, about 20 fold, about 30 fold, and at least about 40 fold) of an immunosuppressive cytokine upon stimulation of T cells from subjects prepared from HSC/HPC transduced with TCR described herein compared control T cells (vector transduced).

The phrase “conditioned for transplantation” as used herein, refers to the treatment of the recipient subject with radiation or cytotoxic agents prior to transplantation. For example, in the methods herein mice are conditioned with irradiation at 900 rad at 4 weeks of age. A milder procedure of conditioning used herein includes irradiating twice with 450 rad at one day interval. An alternative and milder conditioning procedure uses a combination of cytotoxic agents, for example a combination of BCNU (carmustine), etoposide, cytosine arabinoside, and melphalan (BEAM), or the cytotoxic agent Busulfan (Myleran, GlaxoSmithKline) or Busulfex IV (PDL BioPharma, Inc., NV, U.S.A.).

The word “transducing” as used herein refers to delivering a particular gene of interest, for example a gene including the nucleotide sequences of T cell receptor α and J pair polypeptides, to target cells, for example, hematopoietic stem and progenitor cells.

The phrase “regulatory T cell line” as used herein refers to one of a family of cytokine secreting T cells, including Tr1 and Th3 cells, which mediate bystander immunosuppression. (Zhang et al. 2009 Proc. Natl. Acad. Sci USA 106:3336-3341). The regulatory T cell lines used herein to obtain the genes for T cell receptor α and β pair polypeptides are Tr1 like regulatory T cells. Tr1 cells are cytokine-secreting antigen-nonspecific regulatory T cells and mediate bystander immunosuppression. The regulatory T cell lines used herein proliferate specifically in response to stimulation with the immunizing copolymer and also proliferate in varying degrees to stimulation by structurally similar copolymers.

The phrase “in vivo cleavable peptide sequence” as used herein refers to a peptide sequence, for example, porcine teschovirus-1 (P2A) peptide sequence ATNFSLLKQAGDVEENPGP (SEQ ID NO: 5), that describes a peptide which is cleaved spontaneously in vivo (inside a cell). The insertion of the nucleotide sequence corresponding to the in vivo cleavable peptide sequence in between the nucleotide sequences of a set of T cell receptor α and β pair polypeptides, as described herein, results in spontaneous in vivo cleavage of the cleavable peptide to yield equal amounts of separated T cell receptor α and β polypeptides that assemble into a T cell receptor.

The phrase of “isolating” bone marrow cells and “enriching” the cells for the presence of hematopoietic stem and progenitor cells, as used herein refer to reducing frequency or depleting those cells that are mature, such as T cells, B cells, monocytes/macrophages, granulocytes, erythrocytes and their committed precursors such as erythrocyte. Such depletion is performed by using for example, MACS Lineage Cell Depletion Kit, (Miltenyi Biotec, Cambridge, Mass.).

The term “stimulation” as used herein refers to contacting a T regulatory cell line with a peptidic composition, for example a random amino acid copolymer poly (Y,F,A,K)ₙ also referred to as YFAK or FYAK multiple times, for example three times at a defined intervals, for example two weeks, in the presence of one or more cytokine, for example, interleukin-2. (Zhang et al. 2009, Proc. Natl. Acad. Sci. USA 106:3336-3341; Stern et al. 2008, Proc. Natl. Acad. Sci. USA 105:5172-5176).

T cell lines are established from human subjects, for example normal humans not affected by an autoimmune disease, and from MS patients, treated with a peptidic composition, for example treated with YFAK, which reduce the symptoms of MS. Blood samples are obtained and T cells of the phenotype CD4+CD25+ are isolated by affinity to a solid phase having a specific antibody or by FACS of labeled cells. The T cells are restimulated in vitro using for example the peptidic composition used in treating the subject to obtain primary T cell lines. The primary T cell lines thus obtained are restimulated, for example, three to four times at two week intervals in the presence of interleukin 2. The T cell lines obtained after multiple stimulations are analyzed for production of high amounts of immunosuppressive cytokine IL-10 or other immunosuppressive cytokines such as IL-4. The T cell receptor Vα and Vβ repertoires of the T cell lines are each characterized using variable region specific antibodies or by quantitative polymerase chain reaction (qPCR). T cells from the T cell lines are sorted by single-cell sorting into 96-well plates to obtain purified individual T cells which secrete a high level of at least one immunosuppressive cytokine. Genes for T cell receptor α and β polypeptides are isolated using PCR and specific primers from the single cells thus obtained. These genes are used for construction of retroviral vectors for transducing hematopoietic stem and progenitor cells, for transplanting into human subjects for treatment of autoimmune diseases.

A skilled person will recognize many suitable variations of the methods to be substituted for or used in addition to those described above and in the claims. It should be understood that the implementation of other variations and modifications of the embodiments of the invention and its various aspects will be apparent to one skilled in the art, and that the invention is not limited by the specific embodiments described herein and in the claims. Therefore, it is contemplated to cover the present embodiments of the invention and any and all modifications, variations, or equivalents that fall within the true spirit and scope of the basic underlying principles disclosed and claimed herein.

The invention now having been fully described above, additional embodiments are found in the claims herein which are exemplary and not to be construed as further limiting. The contents of all literature and patent documents cited herein are hereby incorporated herein by reference.

### EXAMPLES

### Example 1

### Transduction of Hematopoetic Stem and Progenitor Cells (HSC/HPC) with TCR Expressing Retroviruses for Development of IL-10-Secreting Regulatory T Cells In Vitro

HSC/HPC are highly enriched in the c-kit-positive, Sca-1-positive, lineage-marker-negative (KSL) fraction of mouse bone marrow cells. The expression level of CD34 antigen distinguishes hematopoietic stem cells (HSC) from their progeny: long-term multilineage repopulating cells. Common lymphoid progenitors (CLP) are found more frequently in the CD4 negative KSL (CD34− KSL) fraction of the mouse bone marrow, whereas short-term repopulating cells are present in the CD4 positive KSL (CD34+KSL) fraction. Moreover, the proportion of cells that show in vivo T cell differentiation potential among CD34− KSL cells is significantly larger than that among CD34+KSL cells.

Bone marrow was extracted from the femur and tibia of SJL mice and hematopoietic progenitor cells (HPC), containing both HSC and CLP, were prepared using a standard procedure (Ema, H., et al. Curr Protoc Immunol. 2005 Chapter 22: Unit 22B.1). To obtain cells expressing TCR of interest, these HPCs were infected with retroviruses containing four of the TCRαβ pairs shown in FIG. 1 identified by asterisks (TCRs 1-4). The four TCRαβ pairs contained different related CDR3 sequences. Two approaches for retrovirus infection were tested and the procedure that yielded higher infection efficiency as evaluated by GFP expression with flow cytometry (FIG. 2) was used.

### Example 2

### In Vitro Development of IL-10-Secreting Regulatory T Cells

T cell development from HPC normally takes place in the thymus, and a novel alternative involves coculture of HPC with OP9-DL1 cells, which provides a simple and efficient system for the in vitro generation of T-lineage cells (de Pootera, R. et al. Curr Opin Immunol, 2007 19: 163-168; and Dai, B. et al. Stem Cells Dev (2009) 18: 235-245). The OP9-DL cell line is a murine bone marrow-derived stromal cell line retrovirally transduced to express the Notch ligand, Delta-Like-1 (DL1), which particularly benefits the development of T cells. This cell line is capable of supporting the growth and differentiation of multiple hematopoietic cell lineages from hematopoietic stem/progenitor cells of both mouse and man. HSC/HPC transduced with TCRs 1-4 were then seeded onto OP9-DL1 cells, to which recombinant Flt3 ligand and IL-7 were added, for incubation as described (Dai, B. et al. Stem Cells Dev 2009 18: 235-245; van Lent et al. J Immunol. 2007, 179(8): 4959-68).

FACS analysis of CD3+ T cells that developed revealed that 27% were CD4+CD8+, 26% were CD4+, and only 2% were CD8+. The CD4:CD8 ratio was 13:1. Thus, TCR from a CD4+ T cell gave rise selectively to the development of CD4+ T cells from HPC in this system. Moreover, cytokine analysis of supernatants revealed that small amounts of IL-10 (˜150 pg/ml/10⁵ cells) and no IL-4, IL-13, IFN-γ, or IL-2 were detected (FIG. 4). The IL-10 observed was less than that produced under the same conditions by a YFAK-specific T cell line generated from splenocytes (˜1,000 pg/ml/10⁵ cells) (Stern, J. N. et al. Proc Nal Acad Sci USA 2008. 105(13): 5172-76). It was observed that that I-As, the appropriate MHC protein for the selection of these TCRs was not present on OP9-DL cells. The T cells were presumably weakly selected on the allelic MHC II of the OP9-DL1 cells. Consequently, the yield of IL-10 was lower, although highly significant.

### Example 3

### In Vivo Development of IL-10-Secreting Regulatory T Cells in SJL Mice from Transduced HSC/HPC

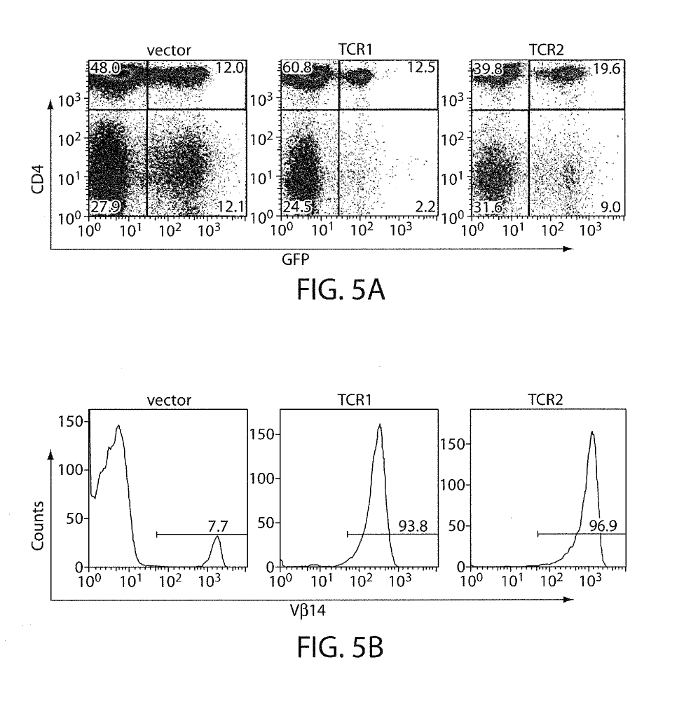

Retrogenic SJL mice were prepared using two of the TCR Vβ14 Vα3.2 pairs that differed only in the CDR3 sequences and identified in IL-10-secreting regulatory T cells induced by the amino acid copolymer YFAK (FIG. 1). These pairs accounted for about 30% of the TCR; Vβ4 TCR pairs that had a more variable TCRα usage accounted for about 24%. In this in vivo example T cells were selected in the thymus on I-A^{s}. HSC/HPC were prepared from the bone marrow of SJL mice as described above and transduced with the retrovirus vector encoding TCR1 or TCR2, two of the TCR Vβ14 Vα3.2 pairs (α β pairs 1 and 7 in FIG. 1), and with an “empty” vector control. The transduction frequency of HSC/HPC measured by GFP expression from the vectors encoding the two TCRs was 54% and 62%, respectively. Then, 1×10⁶ transduced HSC/HPC were injected i.v. into four week old SJL mice that had been conditioned for transplantation by irradiateding with 900 rads to ablate the bone marrow. These mice are referred to as retrogenic mice (Yang, L. et al. Proc Natl Acad Sciences 2002 99(9): 6204-6209; Hoist, J. et al. Nature Methods (2006) 3: 191-197; and Hoist, J. et al. Nature Protocols 2006 1: 406-417). Tail bleeds and FACS to analyze reconstitution 5-6 weeks after transfer indicated that 17% of CD3+CD4+PBMC expressed GFP in the case of TCR1 and 33% in the case of TCR2 (FIG. 5A, upper left and right quadrants). Furthermore, in the case of TCR1 and TCR2, 84% and 68% of the GFP+ cells, respectively, were observed to be CD4+(FIG. 5A, upper and lower right quadrants), and 94% and 97% of the CD4+GFP+ cells, respectively, were observed to be Vβ14 (FIG. 5B).

### Example 4

### Characterization of IL-10 Secreting Regulatory T Cells Developed In Vivo in SJL Mice from HSC/HPC Transduced with TCR Vβ14 Vα3.2

Examples herein have been substantially replicated with minor modifications resulting in greatly improved health of the bone marrow-transplanted retrogenic mice. The irradiation dose of 900 rad was divided into two doses of 450 rad separated by several hours. The period of time for administration of antibiotics prior to and after irradiation at 4 weeks was increased before further experimentation at 12-16 weeks.

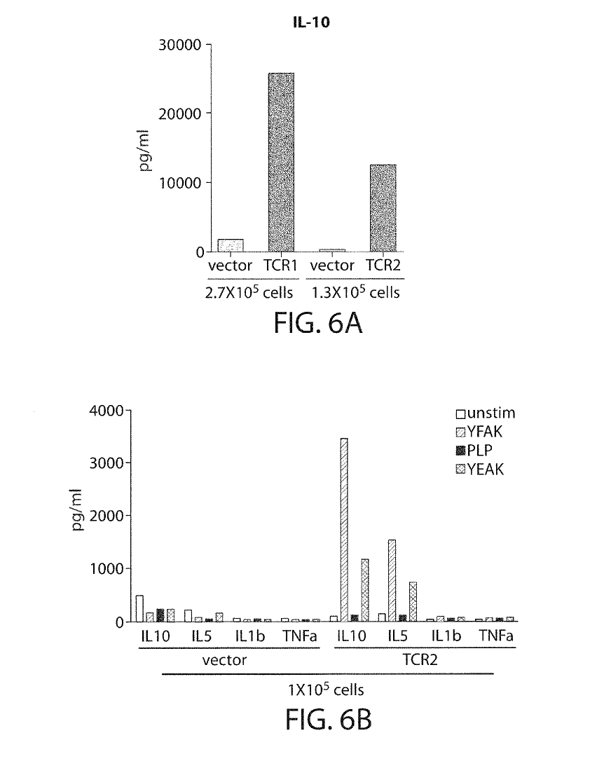

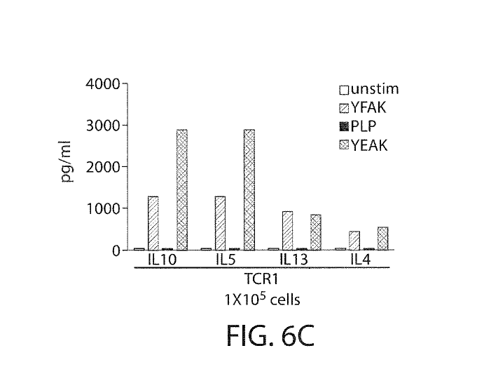

In continuing examples, the IL-10 secreting regulatory T cells in spleens of these retrogenic mice is fully characterized. The frequency of GFP+ CD4+ Vβ14+ T cells in PBMC and splenocytes obtained from these retrogenic SJL mice, expected to be in the range of 15-30% of T cells, is measured, see FIG. 5. IL-10 secretion from these splenocytes stimulated with YFAK is determined, as in FIG. 6, and, in addition, the secretion of IL-2, IL-3, IL-4, IL-5, IL-13, and IFN-γ, before and after stimulation with 10 and 100 μg/ml of YFAK is measured. IL-4 and IL-13 were reported to be secreted by YFAK-specific T cell lines previously (Stern, J. N., et al. Proc Natl Acad Sci USA 2008 105(13): 5172-76. PMCID: PMC2278190.). These splenocytes also secrete IL-5 in addition (FIG. 6). Surprisingly, the quartet of Th2 anti-inflammatory cytokines, IL-4, IL-5, IL-10, and IL-13, is characteristic of the protective response in-duced by helminth infection (Hoffmann, K. F. et al. Adv. Parasitol. 2002 52: 265-307). Splenocytes from YFAK treated mice also secrete IL-3 (Kovalchin, J et al. 2011 PLoS One 6(12): e26274), and it has not been established whether the cells that secrete IL-3 are the same as those that secrete IL-10, IL-4, IL-5, and IL-13. The secretion of one or several cytokines by a single cell population is examined by intracellular cytokine staining as in FIG. 1E of Zhang, H. et al, 2009)

For quantitative assays, splenocytes are stimulated in vitro and cytokines measured using the multi-plex Luminex system of specific antibody-coated beads. The expression of GFP from the vector employed (FIG. 2) is used to determine the frequency of these T cells. Serum levels of IL-10 (and of additional cytokines, if warranted) are assessed before and after administration of 50 □g of YFAK to these retrogenic mice. The methods to be used for measuring cytokine secretion from splenocytes and to measure serum levels of cytokines have been described (Kovalchin, J et al. 2011 PLoS One 6(12): e26274; Li, C. et al. Proc Natl Acad Sci 2009 106(14):5767-7).

GFP+CD4+Vβ314+ T cells are purified from splenocytes by FACS sorting and/or MACS. The complete phenotype of these T cells developed from transduced HSC/HPC is compared to that previously reported for YFAK-specific IL-10-secreting regulatory T cell lines developed from mice treated with YFAK (Stern, J. N., et al. Proc Natl Acad Sci USA, 2008. 105(13): 5172-76. PMCID: PMC2278190). Foxp3 expression, which is predicted to be absent since it has been absent in all related lines tested to date is measured. Expression of GARP (LRRG32) and neuropilin, recently described as specific markers for Foxp3+ T regs (Weiss, J. M. et al. J Exp Med 2012 209(10):1723-42; Tran, D. Q et al. Proc Natl Acad Sci USA, 2009 106(32):13445-50), are examined. TCR α chain usage is determined using the specific Vα3.2 mAb, and the number of cells reacting with the Vα3.2 mAb is substantially smaller than the number reacting with the Vβ14 mAb, usage of additional TCR α chains will be assessed by PCR (Zhang, H. et al. Proc Natl Acad Sci USA, 2009. 106(9): 3336-41). The usage of additional TCR α chains is precluded by allelic exclusion. However, since that does not apply to TCR α chains, additional TCR α chains could rearrange in the developing HSC/HPC, some of which may pair with TCR Vβ14.

The dose response curve for secretion of IL-10 after stimulation with YFAK in the range of 1-100 μg/ml is measured. Similarly, the stimulation of IL-10 secretion by anti-CD3 mAb will be studied. Importantly, the T cell line previously studied expressed a high level of GITR (glucocorticoid-induced TNF receptor superfamily member 18 (TNFRSF18) (Stern, J. N. et al. Proc Natl Acad Sci USA, 2008. 105(13): 5172-76; Watts, T. H. Annu Rev Immunol. 2005; 23:23-68). Its ligand, GITRL (TNF superfamily member 18 (TNFSF18)) (Watts, T. H. Ibid.; Azuma, M. Crit Rev Immunol. 2010 30(6):547-57), has been reported to exist on microglia and on plasmacytoid dendritic cells (Hwang, H. et al. J Neurosci Res. 2010 88(10):2188-96; Hanabuchi, S. et al. Blood (2006) 107(9):3617-23) and to costimulate the secretion of IL-10 resulting in production of large amounts of IL-10 [38]. Consequently, the effect of GITRL on IL-10 production resulting from stimulation with limiting amounts of either YFAK or anti-CD3 is analyzed. This system may have high relevance in the treatment of EAE, as will be described below. The murine GITRL dimer has been produced for X-ray structure determination (Chattopadhyay, K. et al. Acta Crystallogr D Biol Crystallogr. 2009 65(Pt 5):434-9) from Dr. Steven Almo of Einstein Medical School.

### Example 5

### Examination of the Resistance of Retrogenic Mice Expressing Graded Levels of TCR Vβ14 Vα3.2 to Autoimmune Diseases

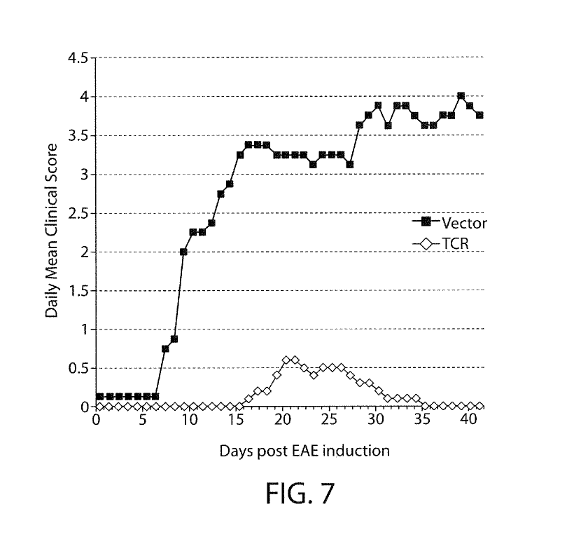

To determine whether overexpression of IL-10 secreting T cells in retrogenic mice protect them from induction of EAE, retrogenic mice expressing GFP+Vβ14+CD4+ T cells as ˜1% of total T cells, the highest level expected in mice treated with YFAK (although the level of IL-10 secreting T cells has never been measured in any circumstance) were produced (FIG. 7). The protocol described herein was modified by diluting the HSC/HPC transduced with TCR Vβ14 Vα3.2 with normal bone marrow from SJL mice. For comparison, the highest level of virus-specific T cells produced during chronic viral infection is in the range of 0.1%-1%.

To extend and validate the result shown in FIG. 7, the susceptibility of retrogenic mice expressing graded levels of TCR Vβ14 Vα3.2 to PLP-139-151-induced EAE is examined. A 1:10 and 1:30 dilution of the transduced HSC/HPC with normal bone marrow is used which is predicted to produce ˜2% and ˜0.7% GFP+ splenocytyes (compare FIG. 5). IL-10 secretion by isolated splenocytyes is measured after stimulation with YFAK and is expected to produce ˜1,000 and ˜333 pg/ml/105 cells (compare FIG. 6). The mice are injected with 100 μg of PLP-139-151 in CFA s.c. at Day 0 followed by 200 ng of pertussis toxin i.v. at Day 1, conditions that induce EAE in control SJL mice, initiating disease at Days 9-11 and reaching a score of 5 (death) at Days 12-14 indicating a very severe disease. Mice are used with and without injection of 50 μg of YFAK at Day −1. Adoptive transfer of 1×10⁶ IL-10-secreting regulatory T cells into SJL mice protected from EAE without stimulation of IL-10 secretion by YFAK (Stem, J. N. et al. Proc Natl Acad Sci USA, 2008. 105(13): 5172-76. PMCID: PMC2278190). In this situation, an endogenous signal induces IL-10 from the adoptively transferred IL-10-secreting regulatory T cells. This signal could be a peptide derived from degradation of any myelin protein during induction of disease, presumably through binding to an innate immune receptor or to the adaptive immune receptor, I-As in SJL mice. The binding to the MHC protein requires only conservation of the character of anchor binding residues (Wucherpfennig, W. F., and Strominger, J. L. Cell 1995 80(5): 686-705) and recognition by the T cell receptor has also recently been shown to accommodate several different peptide sequences (Adams, J. J. et al. Immunity (2011) 35(5): 681-93).

Upregulation of GITRL on CNS microglia or dendritic cells differentiated from them induced by inflammation which, acting through GITR, expressed on IL-10 secreting regulatory T cells, could induce secretion of IL-10 exactly at the site of the inflammatory process as the endogenous signal (Hwang, H. et al. J Neurosci Res. 2010 88(10):2188-96; Illes, Z. et al. Eur J Immunol 2005 35:3683-93). The utilization of a specific localized CNS trigger would explains why Copaxone, which has been in clinical use for several decades, has not led to an increased incidence of severe infections due to generalized immunosuppression. Observations that GITRL expression on dendritic cells can be induced by an inflammatory process, that neutralization of the GITR/GITRL interaction by anti-GITR mAb exacerbates EAE (suggesting that an anti-inflammatory response as well as an inflammatory reaction are induced by immunization) (Kohm, A. P. et al J Immunol. 2004 172(8):4686-90) and that upregulation of GITRL on dendritic cells has been observed in the treatment of multiple sclerosis (Chen, M. et al. J. Neuroimmunol. 2012 242(1-2): 39-46) and are consistent with this model. Expression of GITRL on both myeloid and plasmacytoid DC in the CNS is analyzed before and after induction of EAE using the specific mAb DTA-1 or YGL386 (Kanamaru, F. et al. J Immunol. (2004) 172(12):7306-14; Tone, M. et al. Proc Natl Acad Sci USA 2003 100(25):15059-64.).

These experiments yield estimates of the level of splenic and CNS IL-10-secreting regulatory T cells that protect from the severe disease protocol and whether or not stimulation by a low level of YFAK is required or improves the results. The duration of the resistance will also be assessed by keeping retrogenic mice for 3, 6, and 12 months before testing disease susceptibility. Similar examples are performed inducing EAE in these transplanted SJL mice with 100 μg of MBP-85-99 or inducing EAN with 2.5 mg of bovine peripheral nerve myelin (Stem, J. N. et al. Proc Natl Acad Sci USA, 2008. 105(13): 5172-76).

### Example 6

### In Vitro Stimulation of T Cells Obtained from Retrogenic Mice

Splenocytes were isolated from a TCR1 and a TCR2 mouse, as well as from a vector control mouse. The yield of splenocytes was 2×10⁸ cells from each of vector and TCR1 mouse, and 6×10⁷ cells from the TCR2 mouse that had a smaller spleen. T cells were enriched from the splenocytes using the MACS Pan TCR isolation kit and were then sorted for CD4+GFP+ cells, with yields of 10⁵ to 10⁶ GFP+CD4+ T cells. After stimulation with 50 μg/ml of YFAK, the amount of IL-10 produced was surprisingly high (i.e., approximately 10,000 pg/ml/10⁵ cells in both cases). The vector control cells produced only negligible amounts of IL-10 (FIG. 6A). Compared to vector control cells the amount of IL-10 secreted was about 30 fold higher. The amount was so large that IL-10 precipitated in the culture supernatant. The amount of IL-10 produced by splenocytes from mice treated with YFAK that became resistant to induction of EAE was observed in the range of 1,000 pg/ml/10⁵ cells. The stimulation examples were performed multiple times (three times with TCR1 and four times with TCR2) using a modified procedure. Secretion of anti-inflammatory cytokines IL-10, IL-5, IL-13, IL-4, and of the pro-inflammatory cytokines IL-1β and TNFγ as controls was measured (FIGS. 6B and C). The amount of IL-10 secreted using the modified procedure was 1,000-3,500 pg/ml/10⁵ cells, also a very high level. A similar amount of IL-5 secretion was observed. The amount of IL-13 in one experiment was 900 pg/ml/10⁵ cells and of IL-4 about 500 pg/ml/10⁵ cells.

### Example 7

### Susceptibility of Transplanted SJL Mice to EAE

Initial examples to test susceptibility to PLP 139-151 induced EAE in the transplanted mice were unsuccessful because most of the mice died of “acute toxicity” within 24 hours of receiving the injection of PLP139-151 in Complete Freund's Adjuvant to induce EAE. The first signs of EAE do not appear until 9-10 days after injection. Severe radiation protocols had not provided sufficient time for reconstitution or sufficient antibiotics for protection.

A modified protocol for irradiating mice to ablate bone marrow prior to transplantation with transduced HSC/HPC, compared to the one described in Example 3 was used in this Example because mice produced using the irradiation protocol of Example 3 died of “acute toxicity” within 24 hours of receiving the injection of PLP139-151 in Complete Freund's Adjuvant to induce EAE. In the modified protocol mice were irradiated twice at 4 weeks of age at 450 rad with a separation of a few hours between the irradiations. After irradiation and transplantation with HSC/HPC, disease was induced at 12-16 weeks by injection of PLP139-151 in Complete Freund's Adjuvant to induce EAE compared to injection at 8 weeks of age in earlier examples. The additional time resulted in stable reconstitution of the hematopoietic system, particularly of myeloid cells. In this manner stably reconstituted transplanted mice were produced. Retrogenic mice whose splenocytes expressed ˜1% of TCR Vβ14 Vα3.2 (see below) were used to induce EAE. In this Example, EAE was induced with PLP139-151 at 16 weeks of age (after transplantation at 4 weeks), and, remarkably, only two of the five TCR retrogenic mice developed very weak disease (maximum average score 0.5 and transient after which clinical score was further reduced) and three other mice remained disease free. In contrast, two of the five vector control retrogenic mice died within a few days and two developed severe disease (average score=3.9; FIG. 7). No stimulation of the TCR in vivo by YFAK was required.

### Example 8

### Development of Retrogenic Mice Expressing the TCR of IFN-γ-Secreting Pathogenic 5B6 T Cells

Development of retrogenic mice expressing the TCR of IFN-γ-secreting pathogenic T cells, 5B6 T cells is useful for establishing whether the specification of IL-10 secretion is a unique property of the TCR of IL-10-secreting regulatory T cells, and importantly for obtaining information as to whether the TCR of a T cell may encodes specificity for another cytokine. For this example, the TCR of pathogenic 5B6 T cells that recognizes the autoantigen PLP139-151 (Waldner, H. et al. Proc Natl Acad Sci (2000) 97: 3412-7) is used. The pathogenic 5B6 transgenic T cells secrete IL-2 and IFN-γ in response to PLP-139-151 presented by I-As. Splenocytes needed for cloning TCR Vα4 and Vβ6 chain cDNAs, the TCR chains used by the pathogenic 5B6 T cells, are obtained from 5B6 mice (Getts, D. R. et al. Nat Biotechnol. 2012, 30(12):1217-24). The TCR Vα4 and Vβ6 chain cDNAs are inserted into the retrovirus vector shown in FIG. 2. This vector is used to transduce HSC/HPC and to create retrogenic mice expressing a high level of the T cells expressing the 5B6 TCR as described above and in Zhang et al 2009. The splenoctyes of these retrogenic mice are examined for secretion of the cytokines IL-2, IFN-γ, and IL-10 with and without stimulation by 10 and 100 γg/ml PLP-139-151. In addition, their susceptibility to the induction of EAE by PLP-139-151 and the development of EAE spontaneously (Waldner, H. et al. Proc Natl Acad Sci 2000 97: 3412-7) are examined.

Splenoctyes from the resulting mice may produce no IL-10 if the phenomenon is limited to the TCR of IL-10-secreting regulatory T cells. Alternatively T cells may induce self-regulation (Kumar V. et al. Intl Rev Immunol. 2005 24: 199-209), and the TCR of pathogenic 5B6 T cells may induce IL-10 secretion, or secrete IFN- and/or IL-2 without or with stimulation by PLP139-151 (and therefore have pathogenic characteristics). Notably, somatic cell nuclear transfer from IFN-γ secreting T cells into ES cells resulted in development of T cells that secrete IFN-γ (Kirak, O., Science, 2010 328(5975): p. 243-8). The property of secretion of IL-10 secretion (and, possibly, other cytokines) by a TCR is a newly discovered function for the TCR molecule. The transgenic T cells of the 5B6 transgenic mice amounted to at least 98% of the T cells in the spleen and secreted both IL-2 and IFN-γ (Waldner, H. et al. Proc Natl Acad Sci 2000, 97: 3412-7). It is therefore envisioned that in the retrogenic mice, the fraction of 5B6 T cells in PBMC and spleen is very high, possibly as high as 50%, and that both IL-2 and IFN-γ are produced.

### Example 9

### Role of a Specific Peptide Encoded within TCR Vβ14 Vα3.2 and Role of Proteolysis of TCR Vβ14 Vα3.2 in Generation of the YFAK-Specific IL-10-Secreting Regulatory T Cells

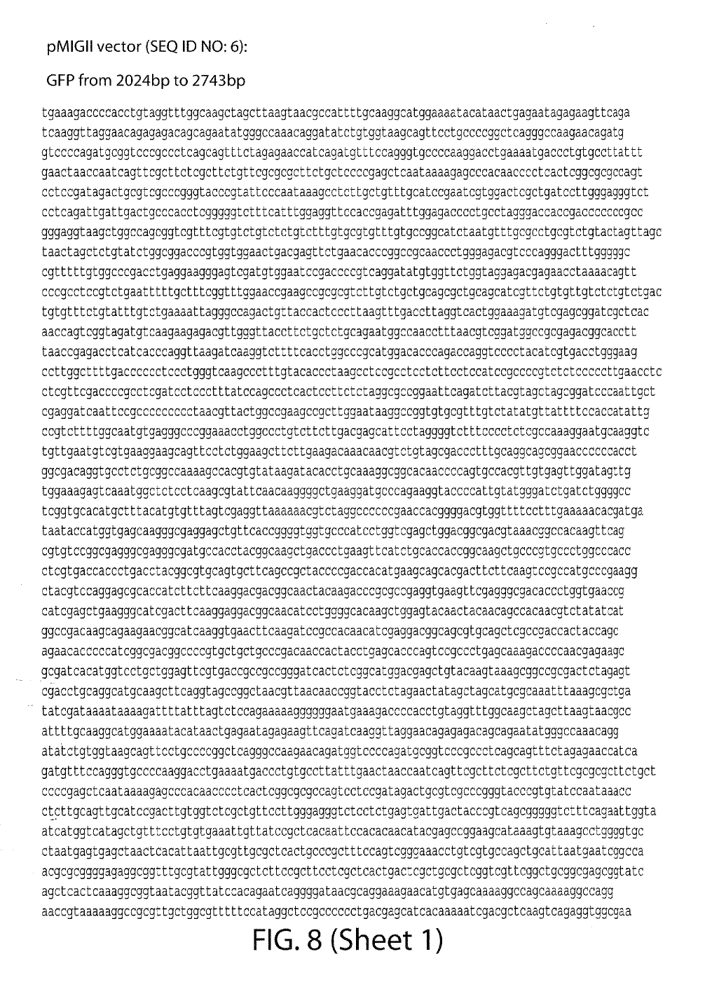

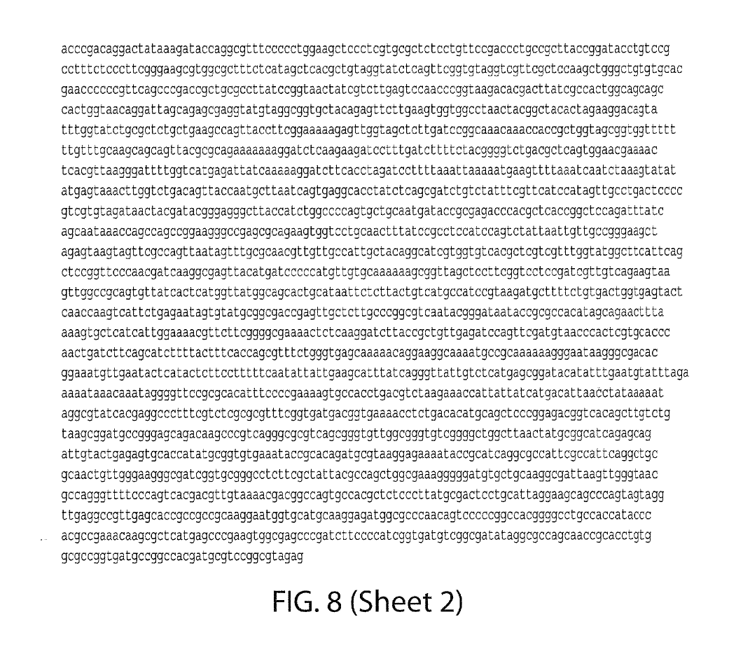

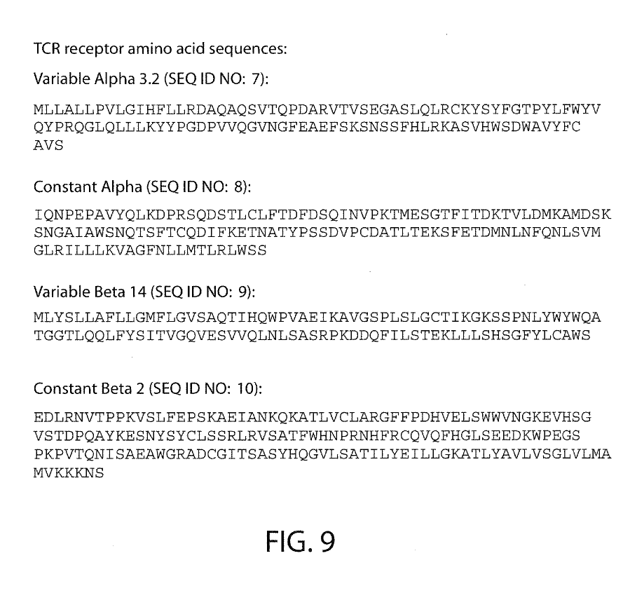

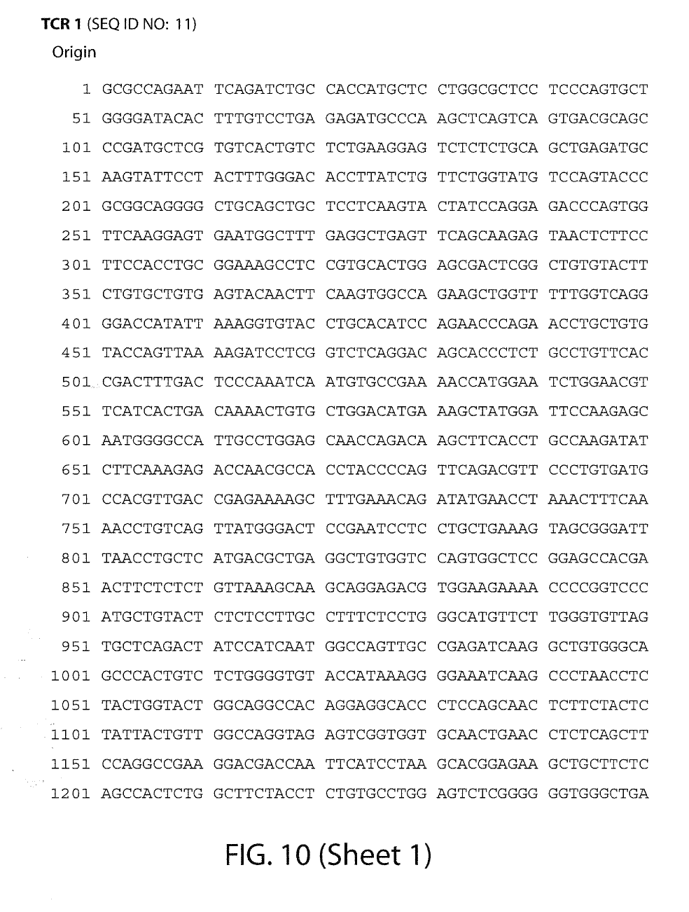

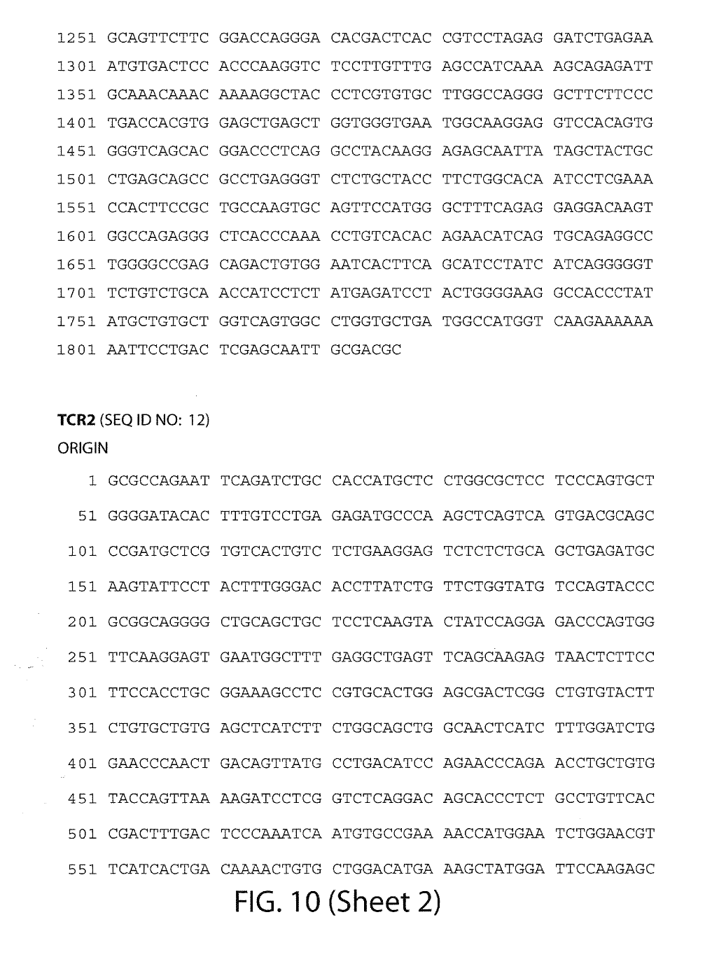

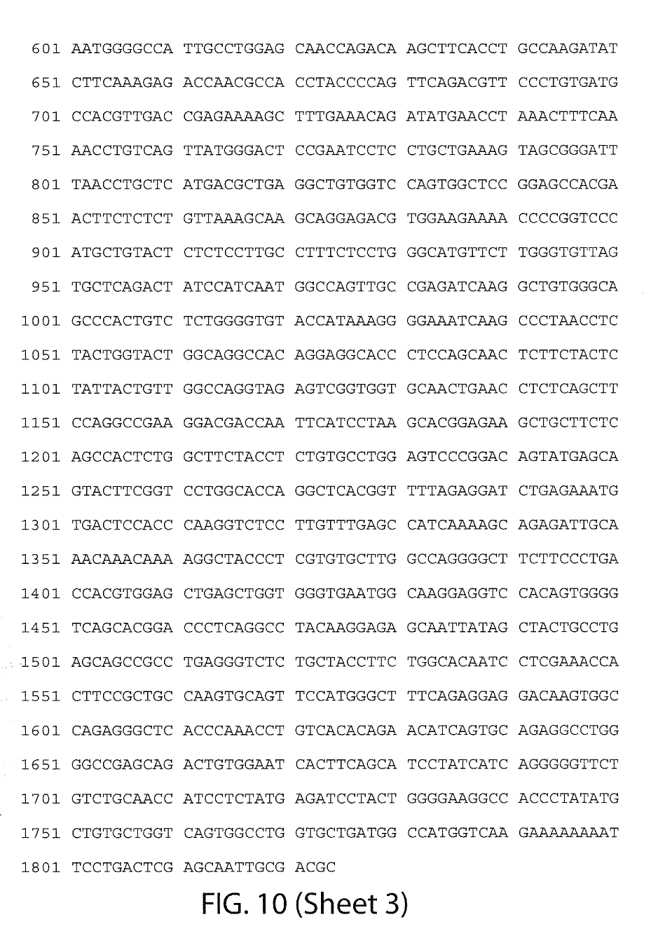

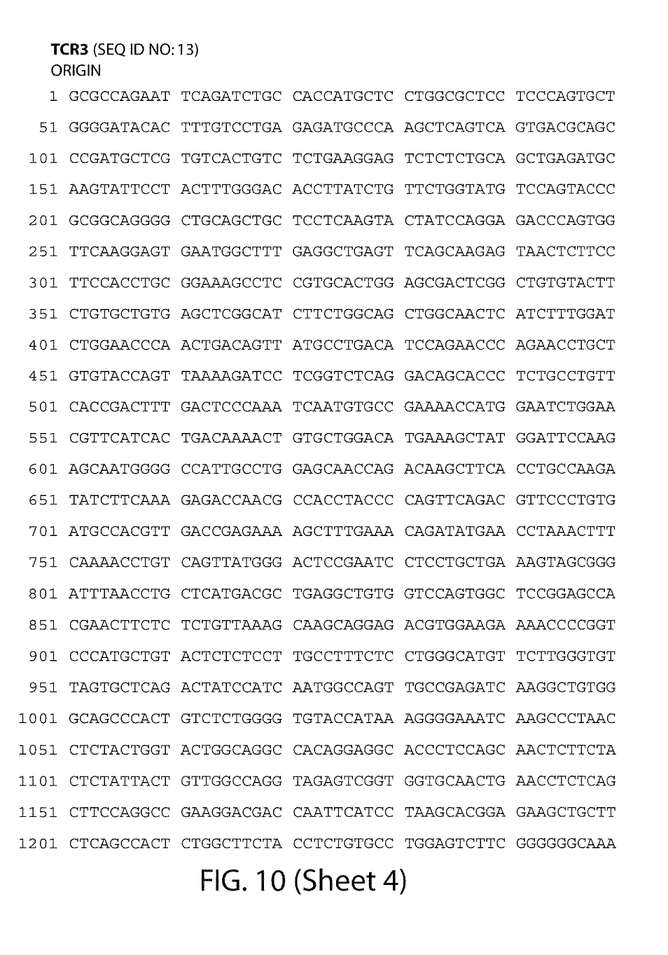

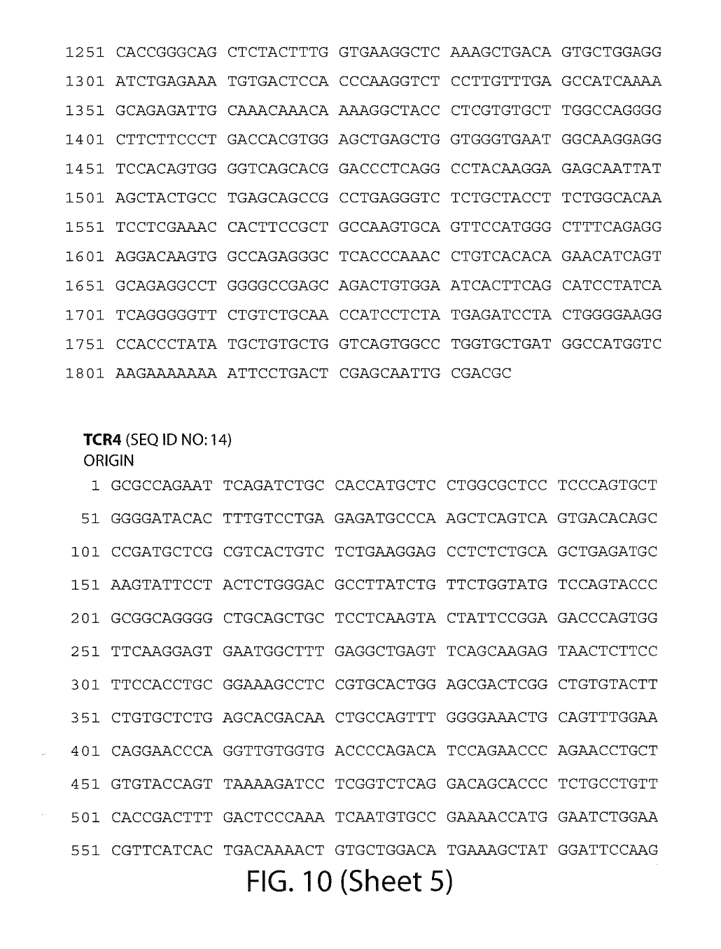

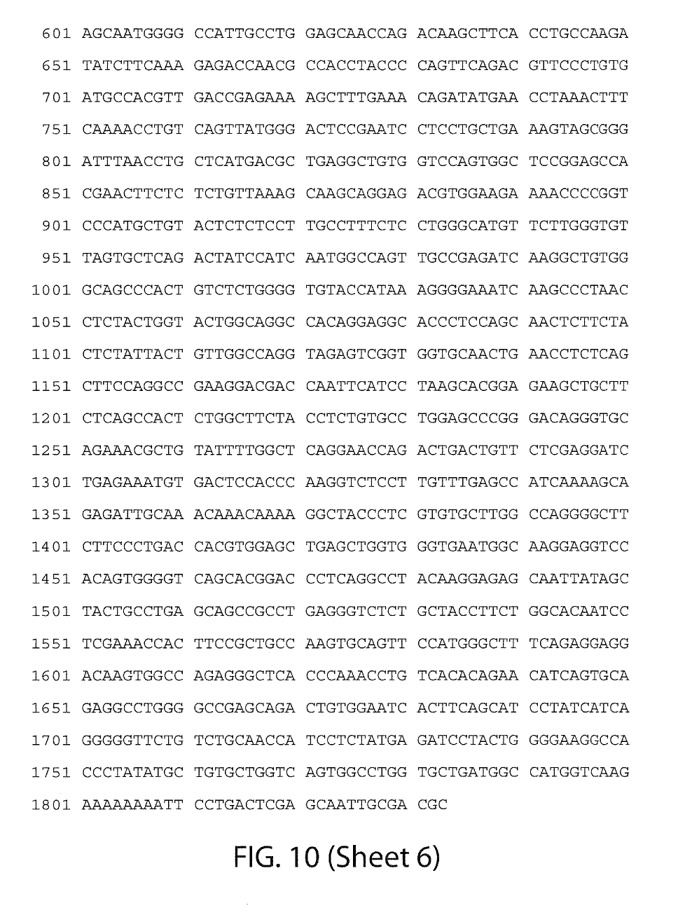

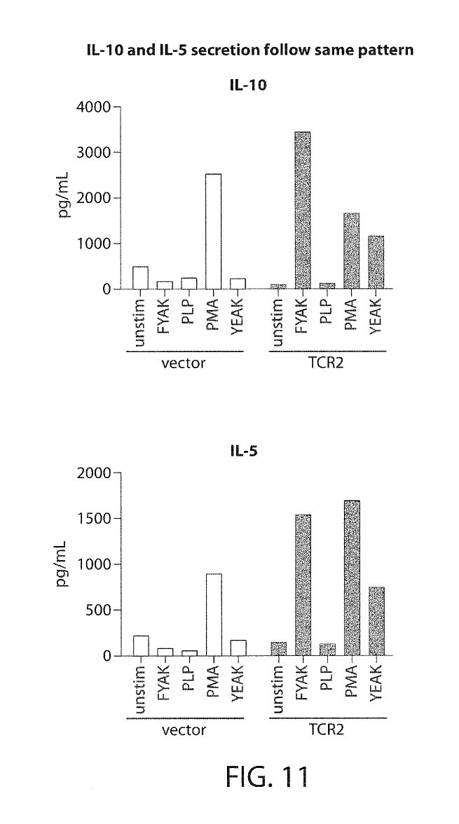

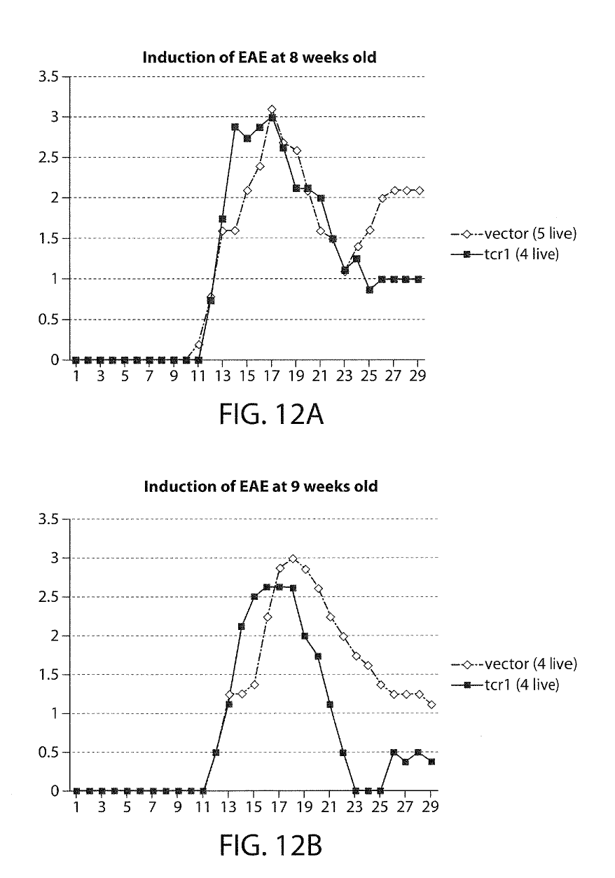

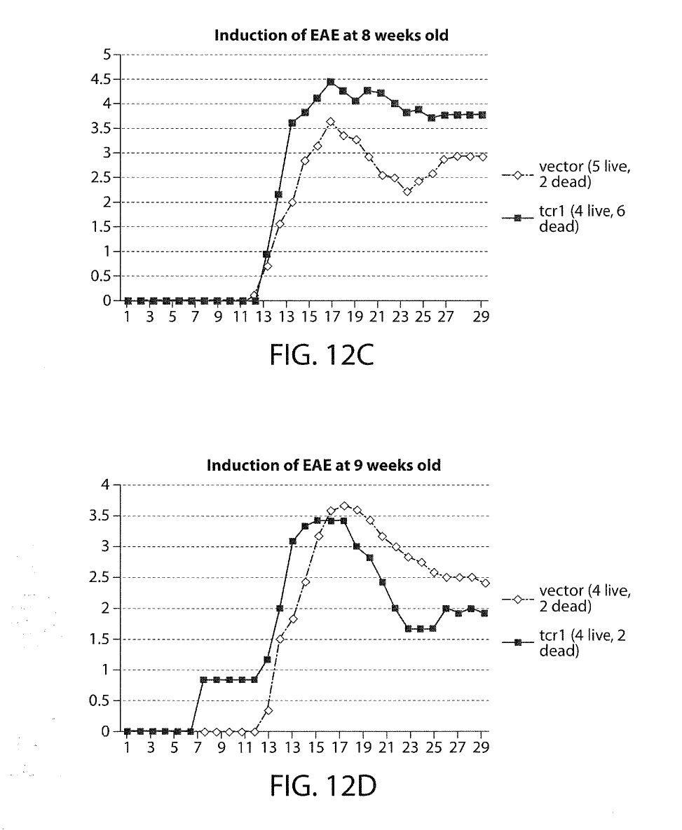

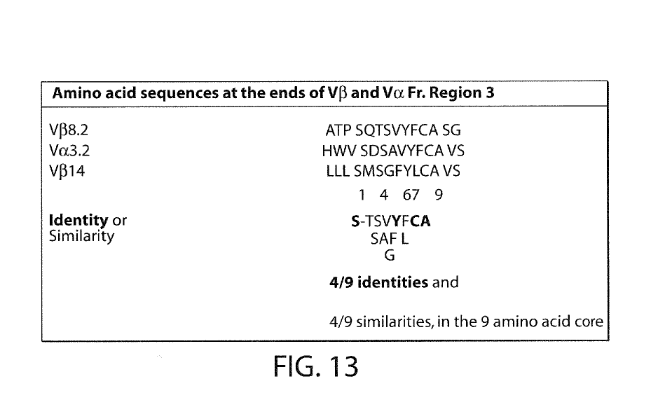

The J5 peptide (Stern, J. N et al. Proc Natl Acad Sci USA, 2005 102(5): 1620-25. PMCID: PMC547868), the S. japonicum peptide (Wang, X. et al. Eur J Immunol. 2009 39(11): 3052-65), and, importantly, a peptide from murine Vβ8.2 (Kumar, V. et al. J Immunol, 1995 154(4): p. 1941-50). can all protect against EAE and the first two have been shown to induce cells that secrete IL-10. The latter peptide is located at the end of the framework region 3 of the Vβ8.2 gene immediately preceding the CDR3 region. The corresponding peptide in the Vβ14 gene and that in the Vα3.2 gene are highly homologous (FIG. 8). The present example establishes whether the Vβ8.2 TCR peptide and the homologous peptides in Vβ14 and Vβ3.2 can induce IL-10 secreting regulatory T cells.

Interestingly, the 9 amino acid core of these three peptides all fit a possible motif for binding to I-A^{s}, with anchor residues P1(S), P4(S, A, G), P6(Y), P7(F, L), and P9(A) (Kalbus, M., et al. Eur J Immunol (2001) 31: 551-562), Three of their amino acids at P1, P6, and P9 are identical in these three sequences and two, P4 and P7, highly similar. Their T cell contact sites at P3 and P5 are also similar. Three peptides are synthesized, and ability of each to induce IL-10-secreting regulatory T cells and protect from EAE is examined using the same procedure used with the J5 peptide (Stern, J. N. et al. Proc Natl Acad Sci USA, 2005 102(5): 1620-25. PMCID: PMC547868).

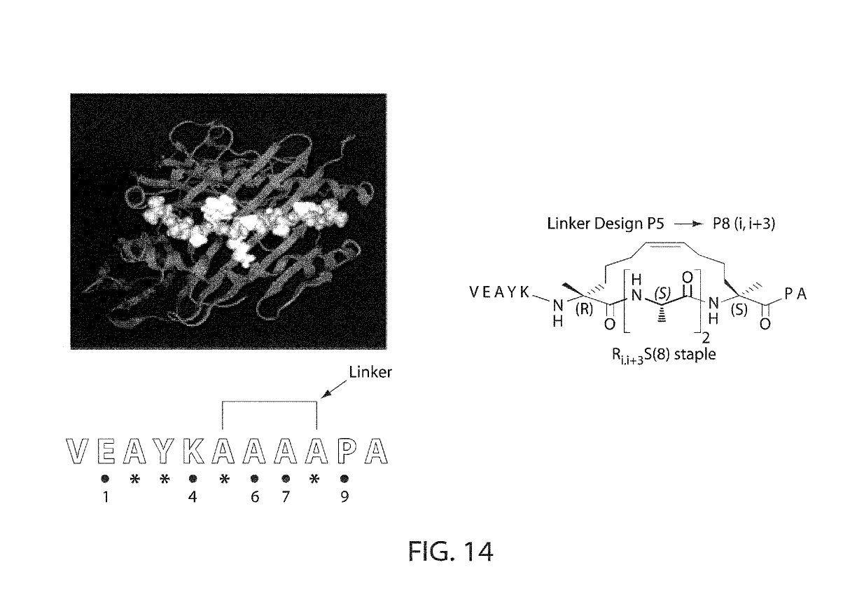

A further development would be to modify the peptide to enhance its affinity for I-A^{s}, and, even more interesting, to staple the peptide using all-hydrocarbon i, i+3 staples to create a peptide with a polyproline-II-like helix (Walensky, L. D. et al. Science 2004 305(5689): 1466-70; Kim, Y. W. et al. Org Lett. 2010 12(13): 3046-9; Kim, Y. W. et al. Nat Protoc. 2011 6(6): 761-71; and Bird, G. H Proc Natl Acad Sci. 2010 107(32): 14093-8). See FIG. 14. So far as is known, all peptides that bind to MHC Class II proteins adopt this helical configuration (Jardetzky, T. S. et al. Proc Natl Acad Sci. (1996) 93(2): 734-738. PMCID: PMC40123). The fixation of peptides in this configuration by stapling should greatly enhance the binding as well as provide protection from proteolysis; particularly in this case, from a chymotrypsin-like activity present in serum. Moreover, it has been reported that such stapled peptides may be orally active (Bird, G. H Proc Natl Acad Sci. 2010 107(32): 14093-8). To form the staple the unnatural amino acid α-methyl, α-pentenyl glycine with R-configuration at P5 and S-configuration is substituted at P8 (Kim, Y. W. et al. Org Lett. 2010 12(13): 3046-9), of any identified peptide with the desired activity, followed by ring closing olefin metathesis to create the staple. Alternative stapling positions (e.g., P6 and P9, P7 and P10, or P8 and P11), configurations (e.g., S₅, S₈, or R₅, R₈), and stapling lengths are evaluated. The reagents are available in our Chemistry Department where this technology was developed, as well as commercially.

Thus, this approach has the potential to create novel, orally available peptides with therapeutic activity against EAE (and, by extension, against MS). These examples validate and extend prior work by identifying the regulatory T cells involved. A nested set of peptide 15mers covering the Vβ14 gene are synthesized and tested as described (Cuddapah, S. et al. Curr Opin Immunol. 2010 22(3): 341-7). Finally, the techniques applied above are used to study peptides from the Vα4Vβ6 TCR used to generate the 5B6 transgenic mice (Waldner, H. et al. Proc Natl Acad Sci (2000) 97: 3412-7). No information is presently available to provide clues to the mechanism by which this TCR specifies IFN-γ and IL-2 secretion, if it does so, as suggested by ref. (Kirak, O. et al. Science, 2010 328(5975): p. 243-8).

### Example 10

### Proteolysis-Defective CD3 Subunits to Assemble TCR Vβ14 Vα3.2 in Retrogenic Mice to Elucidate the Mechanism of Induction of IL-10 Secretion

TCR development is a complex process. TCR “double negative” (DN) thymocytes develop first followed by CD4+CD8+“double positive” (DP) thymocytes. One of the co-receptors is then lost and the cells become CD4+ or CD8+“single positive” thymocytes. A striking feature of this developmental process is that the surface expression level of the TCR with its four CD3 subunits is high on DN thymocytes but is very low on DP thymocytes and then much higher, perhaps ten times higher, on single positive thymocytes and peripheral T cells. This change in expression level functions in the process of T cell selection in the thymus by MHC proteins (Bluestone, J. A. et al. Nature 1987 326: 82-4; Daniels, M. A. et al. Nature 2006 444: 724-9) or for other undiscovered reasons. A recent study of this phenomenon led to the conclusion that proteolysis of the TCR in the double positive, but not in the single positive, thymocyte is responsible for the change in surface expression (Wang, H. et al. EMBO J 2010 29: 1285-1298). The messenger RNA levels for the TCR chains and for the four CD3 subunits were observed to be the same in single and double positive thymocytes. Ubiquitination followed by degradation in lysosomes was found to be responsible for degradation of the TCR/CD3 complex at the DP stage. A mutant CD3-γδεζ vector in which all 37 of the lysines (potential ubiquitination sites) in the four CD3 subunits were mutated to arginines was created and introduced into a CD3 null mouse by retroviral transduction of stem cells (the CD3-ζ KO mouse is CD3 null because its CD3-γ and CD3-δ genes cannot be expressed). Restoration of the surface level of the TCR/CD3 complex in double positive thymocytes was achieved. Moreover, when a CD3-ζ-monoubiquitin fusion complex was introduced into the mutated CD3 null mouse, a significant reduction of the high expression level of the TCR/CD3 complex on double positive thymocytes occurred. These mutants are used to investigate the role of TCR proteolysis in the generation of IL-10-secreting regulatory T cells.

HSC/HPC from SJL mice are co-transduced with the vectors encoding TCR Vβ14 Vα3.2 and that encoding CD3-γδεζ in which all 37 lysine residues have been mutated to arginine. The mutant CD3 subunits are transferred to a lentivirus vector that also encodes puromycin resistance. The lentivirus vector will be used to insure that the frequency of transduction is greater than 95%. Then, HSC/HPC are infected with the lentivirus. Two days later the medium is changed. Transduction with the retrovirus encoding TCR Vβ14 Vα3.2 and GFP are carried out in a medium containing puromycin for two additional days. Selection is based on GFP expression from the retrovirus vector containing TCR Vβ14 Vα3.2, as described herein. The transduced GFP+HSC/HPC are used to reconstitute irradiated mice, also as described herein. The T cells from the thymus of these mice as well as from the spleen are analyzed for frequency of single and double positive GFP+ T cells. The success of the procedure is assessed by examining the surface expression level of the TCR in thymocytes and splenocytes. If proteolysis is blocked, then the TCR of the double positive thymocytes is envisioned to be at the same level of surface expression as that found in single positive thymocytes and in splenocytes, i.e., ˜10 times that found in normal double positive thymocytes. Success will depend on whether the high level expression of the mutant CD3 subunits from the lentivirus vector will outcompete the expression from the endogenous CD3 subunits to assemble with the TCR in the double positive thymocytes. The level of IL-10 secretion of splenocytes after stimulation with YFAK is measured and is low if proteolysis is required for the induction of IL-10-secreting regulatory T cells.

Mice have been produced in which none of the four CD3 subunits is expressed, termed CD3-εζ ko mice ((Wang, H. et al. EMBO J 2010 29: 1285-1298). In these mice, the CD3-ε and CD3-ζ chains have been deleted, and the CD3-γ and CD3-δ genes are silenced as a result of insertion of the neo cassette into the CD3-ε gene. However, these mice are in the C57Bl/6 background, expressing H-2^{b}. In order to obtain the I-A^{s} background for selection of TCR Vβ14 Vα3.2, the CD3 mutant mice are backcrossed to SJL mice, for at least 8-14 generations, requiring more than a year of breeding. The desired progeny are selected by the absence of CD3ε and CD3ζ in tail snips assessed by PCR. The resultant SJL mice, designated congenic SJL.B6(CD3εζ ko) mice, carrying the mutant CD3 alleles from the B6 strain, are then used in the examples described herein.

Ubiquitination of either CD3-δ, CD3-ε, or CD3-ζ was sufficient to induce proteolysis, but CD3-γ did not participate, indicating functional redundancy in ubiquitination sites. A mono-ubiquitin fusion construct, mutant CD3-ζ with monoubiquitin at the -C terminus (referred to as CD3^{KR}-Ub), was constructed (Wang, H. et al. EMBO J 2010 29: 1285-1298). This construct is used as an additional control in the experiments described above and is envisioned to restore the surface level of TCR in double positive thymocytes to the low wild-type levels and enhance IL-10 secretion by splenocytes.

Without being limited by any particular theory or mechanism of action, it is here envisioned that the TCR derived from IL-10 secreting regulatory T cells encodes information for specific cytokine secretion. Retrogenic mice constructed utilizing the TCR of IL-10 secreting regulatory T cells transduced into HSC/HPC followed by transplantation into irradiated mice developed T cells that secreted only IL-10 and a family of closely related cytokines, IL-5, IL-13, and a small amount of IL-4. This family of cytokines is also induced in mice infected with helminths. In addition to extending this finding of a novel function for a TCR and using it to examine protection from EAE, a major purpose of the examples herein is to ask whether the phenomenon observed is limited to IL-10 secretion or is extended to other cytokines. The latter concept is supported by somatic cell nuclear transfer from IFN-γ secreting T cells to ES cells. Mice that were developed from these ES cells provided T cells that secreted only IFN-γ (Kirak, O. et al. Science, 2010. 328(5975): p. 243-8). A further purpose is to develop information regarding the mechanism by which the TCR of IL-10 secreting regulatory T cells specifies specific cytokine secretion. It is envisioned that proteolysis of this TCR results in formation of a peptide that induces the regulatory T cells.

Novel therapies are examined for EAE, the best mouse model of the human disease MS. Two related approaches to the therapy of EAE are involved, both involving the generation in vivo of IL-10-secreting regulatory T cells. In one approach, the frequency of regulatory T cells is enhanced by transplantation of hematopoietic stem/progenitor cells transduced with the TCR obtained from IL-10 secreting regulatory T cells induced by the random amino acid copolymer YFAK. The splenocytes of these mice secrete a large amount of IL-10. Protection from EAE is accordingly provided during the lifespan of the mouse without or with further injection of small amounts of YFAK. Patients with MS have been shown to have a functional defect in Tregs (Costantino, C. M. et al. J Clin Immunol. 2008 28(6):697-706). IL-10 secreting regulatory T cells, called Tr1 cells, have been described extensively in humans and have an important role in immunosuppression (Gregori, S. et al. Front Immunol. (2012) 3:30). These techniques are applied to humans with MS to correct the defect in Tregs, principally to humans with malignant forms of MS, such as primary progressive MS or multiple relapses leading to secondary progressive MS for whom no effective therapy is currently available. The TCR from IL-10 secreting regulatory T cells is obtained from patients undergoing amino acid copolymer therapy for MS and is cloned and characterized (or possibly from those already described (Gregori, S. et al. Front Immunol. 2012 3:30) and then tested in mice bearing a human immune system (Brehm, M. A. et al. Blood 2012 119:2778-88). Considerable interest has been evident in using bone marrow transplantation for the treatment of severe cases of MS (Martino, G. Nature Reviews Neurology 2010 6: 247-255; Mancardi, G., and Saccardi, R. Lancet Neurology 2008 7(7):626-36; Reston, J. T. et al. Multiple Sclerosis 2011 17(2):204-13; Freedman, M. S. et al. Multiple Sclerosis 2011 17(2):131-32; Fassas, A. et al. Neurology 2011 76(12):1066-70). The technique described here has the potential to provide lifelong freedom from relapses.

An alternative embodiment for therapy identifies and utilizes naturally occurring peptides derived from the same TCR protein to induce in vivo expansion of Tregs. Without being limited by any particular theory or mechanism of action, it is here envisioned that the naturally occurring materials should be much more potent than the random amino acid copolymers now in clinical use (Copaxone (YEAK) or the more potent copolymer now in Phase II clinical trials (YFAK)). Since these peptides are naturally occurring, potential for complications is much reduced. Substantial evidence already suggests the existence of such peptides, and an important focus of this proposal is to identify them. Chemical modification by stapling improves stability to proteolysis in vivo and could lead to oral availability.

1. A method for treating a subject for an autoimmune disease, comprising:
isolating bone marrow cells from a donor subject and enriching the cells for the presence of hematopoietic stem and progenitor cells (HSC/HPC), and transducing the HSC/HPC by a retroviral vector containing a heterologous gene encoding a set of T cell receptor α and β pair polypeptides linked by an in vivo cleavable peptide sequence, wherein the set of T cell receptor α and β pair polypeptides is obtained from a regulatory T cell line that secretes a high level of at least one cytokine upon stimulation with a peptidic composition that binds to the set of T cell receptor α and β pair polypeptides;
conditioning the recipient for transplantation, and transplanting the transduced HSC/HPC and non-transduced bone marrow cells into a recipient subject having the autoimmune disease; and,
measuring a reduction or an elimination of at least one symptom of the autoimmune disease in the recipient subject, thereby treating the recipient subject for the autoimmune disease.

2. The method according to claim 1, wherein the donor and the recipient subjects are the same subject.

3. The method according to claim 1, wherein the donor and the recipient subjects are different subjects.

4. The method according to claim 1, wherein conditioning for transplantation comprises at least one of irradiating the recipient subject and administering at least one cytotoxic agent to the recipient subject.

5. The method according to claim 1, wherein the subject is an experimental non-human animal and the autoimmune disease comprises experimental allergic encephalomyelitis (EAE), the method further comprising inducing EAE in the recipient after transplantation.

6. The method according to claim 1, wherein the recipient is a human and the autoimmune disease is selected from: multiple sclerosis, autoimmune hemolytic anemia, autoimmune oophoritis, autoimmune thyroiditis, autoimmune uveoretinitis, Crohn's disease, chronic immune thrombocytopenic purpura, colitis, contact sensitivity disease, diabetes mellitus, Grave's disease, Guillain-Barre's syndrome, Hashimoto's disease, idiopathic myxedema, multiple sclerosis, myasthenia gravis, psoriasis, pemphigus vulgaris, rheumatoid arthritis, and systemic lupus erythematosus.

7. The method according to claim 1, wherein the T regulatory cell line secretes a high level of interleukin 10 (IL-10) upon stimulation with a peptidic composition comprising random amino acid copolymer poly(Y,F,A,K)ₙ, also designated YFAK.

8. The method according to claim 7, wherein the T cell receptor α and β pair polypeptides comprise at least one pair of canonical T cell receptor (TCR) variable regions selected form the group consisting of Vα17 and Vβ4, and Vα3.2 and Vβ14, respectively.

9. The method according to claim 8, wherein the variable region Vα3.2 of the T cell receptor α polypeptide comprises a CDR region having at least one amino acid sequence selected from the group consisting of TTSSGQKLV (SEQ ID NO: 1) and at least one Jα region is Jα16 or Jα22; and variable region V β14 of the T cell receptor β polypeptide comprises a CDR region having at least one amino acid sequence selected from the group consisting of LGGWAEQF (SEQ ID NO: 2) and PGQYEQY (SEQ ID NO: 4), and at least one Jβ region selected from Jβ2.1 or Jβ2.7.

10-11. (canceled)

12. A retroviral vector comprising nucleotide sequence encoding a set of T cell receptor α and β pair polypeptides linked by a nucleotide sequence encoding an in vivo cleavable peptide, wherein the nucleotide sequences of the set of T cell receptor α and β pair polypeptides are obtained from a regulatory T cell line that secretes a high level of at least one cytokine upon stimulation with a peptidic composition that binds to the set of T cell receptor α and β pair polypeptides.

13. The retroviral vector according to claim 12, wherein the regulatory T cell line secretes a high level of IL-10 upon binding random amino acid copolymer poly(Y,F,A,K)ₙ.

14. The retroviral vector according to claim 12, wherein the cleavable peptide comprises porcine teschovirus-1 (P2A) peptide comprising amino acid sequence ATNFSLLKQAGDVEENPGP (SEQ ID NO: 5).

15. The retroviral vector according to claim 12, further comprising a nucleotide sequence encoding green fluorescence protein (GFP), wherein the GFP encoding nucleotide sequence is located downstream of nucleotide sequences encoding the set of T cell receptor α and β pair polypeptides, and separated by an internal ribosome entry site (IRES) nucleotide sequence.

16. The retroviral vector according to claim 15, wherein the set of T cell receptor α and β pair polypeptides comprise the canonical TCR variable regions Vα3.2 and Vβ14, respectively.

17. The retroviral vector according to claim 16, wherein the canonical TCR variable region Vα3.2 of the set of T cell receptor α polypeptide comprises a CDR region having amino acid sequence SSSGSWQLI (SEQ ID NO: 3) and the Jα region Jα22, and wherein the canonical TCR variable region V β14 of the T cell receptor β polypeptide comprises a CDR region having amino acid sequence PGQYEQY (SEQ ID NO: 4) and the Jβ region Jβ2.7.

18. (canceled)

19. The retroviral vector according to claim 16, further comprising:
a green fluorescence protein (GFP) encoding nucleotide sequence, wherein the GFP encoding nucleotide sequence is downstream of the set of T cell receptor α and β pair polypeptide encoding nucleotide sequences and separated by an internal ribosome entry site (IRES) nucleotide sequence; and wherein, the set of T cell receptor α and β pair polypeptides comprise the canonical TCR variable regions Vα17 and Vβ4, respectively.

20. A population of transduced hematopoietic stem cells and progenitor cells suitable for engrafting in a host for selectively expressing a desired set of T cell receptor α and β pair polypeptides, the population comprising:
the hematopoietic stem and the progenitor cells transduced with a nucleotide sequence encoding the set of T cell receptor α and β pair polypeptides linked by a nucleotide sequence encoding an in vivo cleavable peptide, wherein the nucleotide sequences encoding the set T cell receptor α and β pair polypeptides are obtained from a regulatory T cell line that secretes high levels of at least one cytokine upon stimulation with a peptidic composition that binds to the T cell receptor α and β pair polypeptides.

21. The population of cells according to claim 20, wherein the peptidic composition comprises random amino acid copolymer poly (Y,F,A,K)ₙ.

22. The population of cells according to claim 20, wherein the cytokine secreted at a high level by the regulatory T cell line upon stimulation with the random amino acid copolymer poly (Y,F,A,K)ₙ is IL-10.

23. The population of cells according to claim 22, wherein the T cell receptor α and β pair polypeptides comprise at least one pair of canonical TCR variable regions selected from the group consisting of Vα3.2 and Vβ14, and Vα17 and Vβ4, respectively.

24. The population of cells according to claim 23, wherein the variable region Vα3.2 of the T cell receptor α polypeptide comprises a CDR region having at least one amino acid sequence selected from the group consisting of TTSSGQKLV (SEQ ID NO: 1) and SSSGSWQLI (SEQ ID NO: 3) and a Jα region is Jα16 or Jα22, and wherein the variable region V β14 of the T cell receptor β polypeptide comprises a CDR region having at least one amino acid sequence selected from the group consisting of LGGWAEQF (SEQ ID NO: 2) and PGQYEQY (SEQ ID NO: 4) and a Jβ region is Jβ2.1 or Jβ2.7.

25-26. (canceled)

27. A method for treating a subject for an autoimmune disease by administering an expanded population of regulatory T cells, the method comprising:
isolating bone marrow cells from a donor subject and enriching the cells for presence of hematopoietic stem cells and progenitor cells (HSC/HPC) and transducing the HSC/HPC with a retroviral vector containing a heterologous gene encoding a set of T cell receptor α and β pair polypeptides linked by an in vivo cleavable peptide sequence, wherein the set of T cell receptor α and β pair polypeptides is encoded by nucleotide sequences obtained from a regulatory T cell line that secretes a high level of at least one cytokine upon stimulation with a peptidic composition that binds to the T cell receptor α and β pair polypeptides;
transplanting the transduced HSC/HPC and non-transduced bone marrow cells into a recipient subject having the autoimmune disease, wherein the recipient is conditioned for transplantation; and,
measuring a reduction or an elimination of at least one symptom of the autoimmune disease in the recipient subject, thereby treating the recipient subject for the autoimmune disease.

28. The method according to claim 27, further comprising after transplanting the transduced HSC/HPC and non-transduced bone marrow cells into the recipient subject having the autoimmune disease and conditioned for transplantation, measuring in the recipient expansion of regulatory T cells expressing the transduced heterologous gene encoding the set of T cell receptor α and β pair polypeptides.


SEQUENCE LISTING (14 sequences)

SEQ ID NO: 1 - protein, 9 aa, Artificial Sequence
  The sequence was designed and synthesized
TTSSGQKLV

SEQ ID NO: 2 - protein, 8 aa, Artificial Sequence
  The sequence was designed and synthesized
LGGWAEQF

SEQ ID NO: 3 - protein, 9 aa, Artificial Sequence
  The sequence was designed and synthesized
SSSGSWQLI

SEQ ID NO: 4 - protein, 7 aa, Artificial Sequence
  The sequence was designed and synthesized
PGQYEQY

SEQ ID NO: 5 - protein, 19 aa, Artificial Sequence
  The sequence was designed and synthesized
ATNFSLLKQAGDVEENPGP

SEQ ID NO: 6 - DNA, 6327 bp, Artificial Sequence
  The sequence was designed and synthesized
tgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtattcccaataaagcctcttgctgtttgcatccgaatcgtggactcgctgatccttgggagggtctcctcagattgattgactgcccacctcgggggtctttcatttggaggttccaccgagatttggagacccctgcctagggaccaccgacccccccgccgggaggtaagctggccagcggtcgtttcgtgtctgtctctgtctttgtgcgtgtttgtgccggcatctaatgtttgcgcctgcgtctgtactagttagctaactagctctgtatctggcggacccgtggtggaactgacgagttctgaacacccggccgcaaccctgggagacgtcccagggactttgggggccgtttttgtggcccgacctgaggaagggagtcgatgtggaatccgaccccgtcaggatatgtggttctggtaggagacgagaacctaaaacagttcccgcctccgtctgaatttttgctttcggtttggaaccgaagccgcgcgtcttgtctgctgcagcgctgcagcatcgttctgtgttgtctctgtctgactgtgtttctgtatttgtctgaaaattagggccagactgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtcccctacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgccggaattcagatcttacgtagctagcggatcccaattgctcgaggatcaattccgccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatgataataccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgactctagagtcgacctgcaggcatgcaagcttcaggtagccggctaacgttaacaaccggtacctctagaactatagctagcatgcgcaaatttaaagcgctgatatcgataaaataaaagattttatttagtctccagaaaaaggggggaatgaaagaccccacctgtaggtttggcaagctagcttaagtaacgccattttgcaaggcatggaaaatacataactgagaatagagaagttcagatcaaggttaggaacagagagacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaaatgaccctgtgccttatttgaactaaccaatcagttcgcttctcgcttctgttcgcgcgcttctgctccccgagctcaataaaagagcccacaacccctcactcggcgcgccagtcctccgatagactgcgtcgcccgggtacccgtgtatccaataaaccctcttgcagttgcatccgacttgtggtctcgctgttccttgggagggtctcctctgagtgattgactacccgtcagcgggggtctttcagaattggtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagag

SEQ ID NO: 7 - protein, 113 aa, Artificial Sequence
  The sequence was designed and synthesized
MLLALLPVLGIHFLLRDAQAQSVTQPDARVTVSEGASLQLRCKYSYFGTPYLFWYVQYPRQGLQLLLKYYPGDPVVQGVNGFEAEFSKSNSSFHLRKASVHWSDWAVYFCAVS

SEQ ID NO: 8 - protein, 136 aa, Artificial Sequence
  The sequence was designed and synthesized
IQNPEPAVYQLKDPRSQDSTLCLFTDFDSQINVPKTMESGTFITDKTVLDMKAMDSKSNGAIAWSNQTSFTCQDIFKETNATYPSSDVPCDATLTEKSFETDMNLNFQNLSVMGLRILLLKVAGFNLLMTLRLWSS

SEQ ID NO: 9 - protein, 111 aa, Artificial Sequence
  The sequence was designed and synthesized
MLYSLLAFLLGMFLGVSAQTIHQWPVAEIKAVGSPLSLGCTIKGKSSPNLYWYWQATGGTLQQLFYSITVGQVESVVQLNLSASRPKDDQFILSTEKLLLSHSGFYLCAWS

SEQ ID NO: 10 - protein, 173 aa, Artificial Sequence
  The sequence was designed and synthesized
EDLRNVTPPKVSLFEPSKAEIANKQKATLVCLARGFFPDHVELSWWVNGKEVHSGVSTDPQAYKESNYSYCLSSRLRVSATFWHNPRNHFRCQVQFHGLSEEDKWPEGSPKPVTQNISAEAWGRADCGITSASYHQGVLSATILYEILLGKATLYAVLVSGLVLMAMVKKKNS

SEQ ID NO: 11 - DNA, 1827 bp, Artificial Sequence
  The sequence was designed and synthesized
gcgccagaattcagatctgccaccatgctcctggcgctcctcccagtgctggggatacactttgtcctgagagatgcccaagctcagtcagtgacgcagcccgatgctcgtgtcactgtctctgaaggagtctctctgcagctgagatgcaagtattcctactttgggacaccttatctgttctggtatgtccagtacccgcggcaggggctgcagctgctcctcaagtactatccaggagacccagtggttcaaggagtgaatggctttgaggctgagttcagcaagagtaactcttccttccacctgcggaaagcctccgtgcactggagcgactcggctgtgtacttctgtgctgtgagtacaacttcaagtggccagaagctggtttttggtcaggggaccatattaaaggtgtacctgcacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtggctccggagccacgaacttctctctgttaaagcaagcaggagacgtggaagaaaaccccggtcccatgctgtactctctccttgcctttctcctgggcatgttcttgggtgttagtgctcagactatccatcaatggccagttgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagcacggagaagctgcttctcagccactctggcttctacctctgtgcctggagtctcggggggtgggctgagcagttcttcggaccagggacacgactcaccgtcctagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctgactcgagcaattgcgacgc

SEQ ID NO: 12 - DNA, 1824 bp, Artificial Sequence
  The sequence was designed and synthesized
gcgccagaattcagatctgccaccatgctcctggcgctcctcccagtgctggggatacactttgtcctgagagatgcccaagctcagtcagtgacgcagcccgatgctcgtgtcactgtctctgaaggagtctctctgcagctgagatgcaagtattcctactttgggacaccttatctgttctggtatgtccagtacccgcggcaggggctgcagctgctcctcaagtactatccaggagacccagtggttcaaggagtgaatggctttgaggctgagttcagcaagagtaactcttccttccacctgcggaaagcctccgtgcactggagcgactcggctgtgtacttctgtgctgtgagctcatcttctggcagctggcaactcatctttggatctggaacccaactgacagttatgcctgacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtggctccggagccacgaacttctctctgttaaagcaagcaggagacgtggaagaaaaccccggtcccatgctgtactctctccttgcctttctcctgggcatgttcttgggtgttagtgctcagactatccatcaatggccagttgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagcacggagaagctgcttctcagccactctggcttctacctctgtgcctggagtcccggacagtatgagcagtacttcggtcctggcaccaggctcacggttttagaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctgactcgagcaattgcgacgc

SEQ ID NO: 13 - DNA, 1836 bp, Artificial Sequence
  The sequence was designed and synthesized
gcgccagaattcagatctgccaccatgctcctggcgctcctcccagtgctggggatacactttgtcctgagagatgcccaagctcagtcagtgacgcagcccgatgctcgtgtcactgtctctgaaggagtctctctgcagctgagatgcaagtattcctactttgggacaccttatctgttctggtatgtccagtacccgcggcaggggctgcagctgctcctcaagtactatccaggagacccagtggttcaaggagtgaatggctttgaggctgagttcagcaagagtaactcttccttccacctgcggaaagcctccgtgcactggagcgactcggctgtgtacttctgtgctgtgagctcggcatcttctggcagctggcaactcatctttggatctggaacccaactgacagttatgcctgacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtggctccggagccacgaacttctctctgttaaagcaagcaggagacgtggaagaaaaccccggtcccatgctgtactctctccttgcctttctcctgggcatgttcttgggtgttagtgctcagactatccatcaatggccagttgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagcacggagaagctgcttctcagccactctggcttctacctctgtgcctggagtcttcggggggcaaacaccgggcagctctactttggtgaaggctcaaagctgacagtgctggaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctgactcgagcaattgcgacgc

SEQ ID NO: 14 - DNA, 1833 bp, Artificial Sequence
  The sequence was designed and synthesized
gcgccagaattcagatctgccaccatgctcctggcgctcctcccagtgctggggatacactttgtcctgagagatgcccaagctcagtcagtgacacagcccgatgctcgcgtcactgtctctgaaggagcctctctgcagctgagatgcaagtattcctactctgggacgccttatctgttctggtatgtccagtacccgcggcaggggctgcagctgctcctcaagtactattccggagacccagtggttcaaggagtgaatggctttgaggctgagttcagcaagagtaactcttccttccacctgcggaaagcctccgtgcactggagcgactcggctgtgtacttctgtgctctgagcacgacaactgccagtttggggaaactgcagtttggaacaggaacccaggttgtggtgaccccagacatccagaacccagaacctgctgtgtaccagttaaaagatcctcggtctcaggacagcaccctctgcctgttcaccgactttgactcccaaatcaatgtgccgaaaaccatggaatctggaacgttcatcactgacaaaactgtgctggacatgaaagctatggattccaagagcaatggggccattgcctggagcaaccagacaagcttcacctgccaagatatcttcaaagagaccaacgccacctaccccagttcagacgttccctgtgatgccacgttgaccgagaaaagctttgaaacagatatgaacctaaactttcaaaacctgtcagttatgggactccgaatcctcctgctgaaagtagcgggatttaacctgctcatgacgctgaggctgtggtccagtggctccggagccacgaacttctctctgttaaagcaagcaggagacgtggaagaaaaccccggtcccatgctgtactctctccttgcctttctcctgggcatgttcttgggtgttagtgctcagactatccatcaatggccagttgccgagatcaaggctgtgggcagcccactgtctctggggtgtaccataaaggggaaatcaagccctaacctctactggtactggcaggccacaggaggcaccctccagcaactcttctactctattactgttggccaggtagagtcggtggtgcaactgaacctctcagcttccaggccgaaggacgaccaattcatcctaagcacggagaagctgcttctcagccactctggcttctacctctgtgcctggagcccgggacagggtgcagaaacgctgtattttggctcaggaaccagactgactgttctcgaggatctgagaaatgtgactccacccaaggtctccttgtttgagccatcaaaagcagagattgcaaacaaacaaaaggctaccctcgtgtgcttggccaggggcttcttccctgaccacgtggagctgagctggtgggtgaatggcaaggaggtccacagtggggtcagcacggaccctcaggcctacaaggagagcaattatagctactgcctgagcagccgcctgagggtctctgctaccttctggcacaatcctcgaaaccacttccgctgccaagtgcagttccatgggctttcagaggaggacaagtggccagagggctcacccaaacctgtcacacagaacatcagtgcagaggcctggggccgagcagactgtggaatcacttcagcatcctatcatcagggggttctgtctgcaaccatcctctatgagatcctactggggaaggccaccctatatgctgtgctggtcagtggcctggtgctgatggccatggtcaagaaaaaaaattcctgactcgagcaattgcgacgc